(19) United States
(12) Patent Application Publication
(10) Pub. No.: US 2024/0207475 A1
(43) Pub. Date: Jun. 27, 2024

(54) CONTINUOUS, REAL-TIME ULTRAVIOLET DISINFECTION OF POPULATED INDOOR SPACES SYSTEMS AND METHODS

(71) Applicants: Stuart Kaler, San Francisco, CA (US); Lorne Whitehead, Vancouver (CA); Joel Pollack, San Jose, CA (US); Eric Kaler, Minneapolis, MN (US); Harold Trease, Blacksburg, VA (US); Bram Sadlik, Vancouver (CA); Christine Martin, Escondido, CA (US); Lynn Trease, Blacksburg, VA (US); John Carpenter, Castro Valley, CA (US); John Gilbert, Pacifica, CA (US)

(72) Inventors: Stuart Kaler, San Francisco, CA (US); Lorne Whitehead, Vancouver (CA); Joel Pollack, San Jose, CA (US); Eric Kaler, Minneapolis, MN (US); Harold Trease, Blacksburg, VA (US); Bram Sadlik, Vancouver (CA); Christine Martin, Escondido, CA (US); Lynn Trease, Blacksburg, VA (US); John Carpenter, Castro Valley, CA (US); John Gilbert, Pacifica, CA (US)

(21) Appl. No.: 17/913,813

(22) PCT Filed: Mar. 23, 2021
(86) PCT No.: PCT/US2021/023546

§ 371 Date: Sep. 22, 2022

Related U.S. Application Data
(60) Provisional application No. 62/993,546, filed on Mar. 23, 2020.
(60) Provisional application No. 63/002,184, filed on Mar. 30, 2020.
(60) Provisional application No. 63/015,468, filed on Apr. 24, 2020.
(60) Provisional application No. 63/019,364, filed on May 3, 2020.
(60) Provisional application No. 63/023,851, filed on May 12, 2020.
(60) Provisional application No. 63/024,506, filed on May 13, 2020.
(60) Provisional application No. 63/029,611, filed on May 25, 2020.
(60) Provisional application No. 63/045,334, filed on Jun. 29, 2020.
(60) Provisional application No. 63/066,919, filed on Aug. 18, 2020.
(60) Provisional application No. 63/086,526, filed on Oct. 1, 2020.
(60) Provisional application No. 63/088,901, filed on Oct. 7, 2020.
(60) Provisional application No. 63/154,098, filed on Feb. 26, 2021.

(51) Int. Cl. A61L 2/24; A61L 2/10

(52) U.S. Cl. CPC A61L 2/24; A61L 2/10; A61L 2202/14; A61L 2202/25

(57) ABSTRACT

Systems and methods are disclosed that disinfect a populated venue/space in real-time by autonomously employing directed disinfecting light/radiation. One embodiment comprises an ultraviolet (UV) light source which may comprise coherent and incoherent light sources that disinfect surfaces of pathogens; a set of sensors collecting data correlating to characteristics of a plurality of objects and events in the space; a controller that receives data from the set of sensors; identifies areas to be disinfected; identifies whether the UV light source has a clear line-of-sight path to irradiate the area; and sends control signals to the light source to control the application of disinfecting light to areas in the space.

### CROSS-REFERENCE TO RELATED APPLICATIONS

This application is a PCT patent application claiming priority to and the benefit of U.S. Provisional Application Nos. (1) 62/993,546 filed Mar. 23, 2020; (2) 63/002,184 filed 03-30-2020; (3) 63/015,468 filed Apr. 24, 2020; (4) 63/019,364 filed May 3, 2020; (5) 63/023,851 filed May 12, 2020; (6) 63/024,506 filed May 13, 2020; (7) 63/029,611 filed May 25, 2020; (8) 63/045,334 filed Jun. 29, 2020; (9) 63/066,919 filed Aug. 18, 2020; (10) 63/086,526 filed 10-01-2020; (11) 63/088,901 filed Oct. 7, 2020; and (12) 63/154,098 filed Feb. 26, 2021—all hereby incorporated herein by reference in its entirety.

### TECHNICAL FIELD

The present application relates to the disinfection of venues/spaces where living beings are located.

### BACKGROUND

UV (and other wavelengths) light is known to kill various forms of germs and bacteria. Such lighting is used to clean tooth brushes, hospital equipment, etc. More recently, robots have been utilized to disinfect hospital rooms by exposing indoor areas with disinfecting light when people are absent.

### DISCLOSURE OF THE PRESENT APPLICATION

Various systems and methods are disclosed that disinfect a venue/space in real-time by autonomously employing directed disinfecting light/radiation. One embodiment comprises a light source which may comprise coherent and incoherent light sources that disinfect surfaces of pathogens; a set of sensors collecting data correlating to characteristics of a plurality of objects and events in the space; a controller that receives data from the set of sensors; identifies areas to be disinfected; and sends control signals to the light source and the one or more reflectors to control the application of disinfecting light to areas in the space.

### BRIEF DESCRIPTION OF THE DRAWINGS

FIG. 1 is a side view drawing of a venue having air space, objects, and patrons with an operating disinfecting system according to an embodiment of the present application;

FIG. 2 is an architecture of a reflective irradiation system according to an embodiment of the present application;

FIGS. 3A-3C depict the architecture of direct irradiation systems according to an embodiment of the present application;

FIG. 4 is an architecture of a multiplexed disinfection system for more than one venue;

FIG. 5 is an architecture of a controller system made in accordance with the principles of the present application;

FIG. 6 is a flow chart of a cleaning protocol according to embodiments of the application;

FIG. 7 is yet another architecture of a controller system made in accordance with the principles of the present application;

FIG. 8 is a flow chart of a process of an object detection protocol according to embodiments of the application;

FIG. 9 is a flow chart of a failsafe protocol according to embodiments of the application;

FIGS. 10A and 10B depict the placement of duty cycle disinfections according to an embodiment of the present application; and

FIGS. 11A and 11B are figures of UV walls made in accordance with the principles of the present application;

FIGS. 12A and 12B are additional figures of UV walls made in accordance with the principles of the present application;

FIG. 13 is an additional figure of UV walls made in accordance with the principles of the present application;

FIGS. 14A and 14B are additional figures of UV walls made in accordance with the principles of the present application;

FIGS. 15A and 15B depict a disinfecting light fixture made in accordance with the principles of the present application;

FIG. 16 depicts the use of UV walls in the context of medical applications.

FIGS. 17 and 18A and 18B are figures depicting UV disinfecting table surfaces for food preparation and other uses.

### DESCRIPTION OF THE VARIOUS EMBODIMENTS

### Introduction

The present application appreciates the need to kill viruses and other undesirable germs and bacteria in various forums that are highly utilized or populated. The present application encompasses sanitizing the ambient air in a school, coffee shop, auditorium, theater, or airplane cabin, for example, by any desired photonic energy (“light”) (e.g., UV-A, UV-B, UV-C, X-ray, microwave, infrared or other wavelengths known to disinfect)—in either coherent (e.g. laser) or incoherent form (or, in some embodiments, a combination of both)—through the ambient air to disinfect it directly. In addition, the system may direct the light to scan, paint or otherwise illuminate often used surfaces, and distributing the light so as to cast a desired footprint for efficient irradiation of the air or surface(s).

In one embodiment, the present application comprises the application of the desired light in an auditorium or other venue, meeting place, hospital rooms or other spaces where disinfection is desired. The direction of the light may be based in part on recognition of where people are and where they are not, and where they are moving to. In one embodiment, this recognition is acquired by a set of sensors placed within the desired space. Such sensors may comprise: cameras, stereo vision, LiDAR, radar, heat sensors, ultrasonic sensors, audio microphones and the like. Data from these sensors are input into one or more controllers (e.g., comprising processors, memory and the like) that are able to autonomously discern human and other living (e.g., dogs, other pets) (hereinafter “people”, “person”) shapes. Their motions may be detected and the system may be able to model and/or calculate future positions.

The system is able to direct the desired light to areas of open air or surfaces in the venue to avoid (or otherwise miss) illuminating people at the moment of application. This may include, for example, recognizing people, calculating distances between the people and recognizing open spaces or specific surfaces. Software to track and predict motion of people are used, for example, in the self-driving car industry may be repurposed for people tracking and predictive motions in a venue.

The light may originate from laser and/or incoherent light sources. In addition, the light may comprise a mix of non-visible light (which tends to be the disinfecting wavelengths) and visible light. The introduction of visible light with non-visible light may be desired, e.g., to show people the location and direction of the non-visible light. This may also be desirable, e.g., to give people a sense that an area has been disinfected, or to give some warning/indication if the system is incorrectly steering the light. In one embodiment, utilized in theaters, auditoriums, etc. where visible light may be a distraction or otherwise not desirable, all wavelengths of the disinfecting medium may be outside the visible spectrum. In one embodiment, the light source is changeable so as to have the capability to inject visible light (e.g., light of a specific wavelengths) to add lighting effects at certain times or moments if desirable. Such programmable light may, for example, be part of a show or motion picture viewed by patrons of the venue, or for yet another example, visibly showing that a table (or other area) is actively being disinfected. It will be appreciated that irradiation may be made in a short burst or bursts. Adjacent lines of sight may be similarly irradiated and the combined beams may overlap in time or space.

The system may have data that identifies surfaces that may require more or less energy for disinfections (e.g., because of the composition of the surface—e.g. cloth vs. wood or the like). This data may be provided to the system either through sensors or by being programmed that data by the owner of the venue. If there is a surface needing more energy, then the system may allocate more energy in a time period or apply same energy over more time to disinfecting such areas. It may be desired that areas of high reflection or specularity may be avoided to prevent irradiation of unintended areas/objects. The present application includes identifying highly reflective or specular areas and determining if unintended objects are in the specular or reflective path and refraining from irradiation until those paths are clear.

The light sources may be included in apparatuses that are configured to direct the light to a plurality of areas (air spaces, surfaces, etc.). Such direction may be performed in any way or combination including electronically, mechanically, electro-mechanically, or micro-mechanically, and may use, reflection, DMD (Digital Micromirror Device), MEMS (Micro Electro-Mechanical System), diffraction, LCoS (Liquid System on Silicon), mechanical systems including gears, micro-mechanically directed interference patterns, tracks, hinges, etc., among other methods to direct the light to one or more surfaces at appropriate times.

Such systems may include light sources illuminating one or more DMD devices or other types of projection devices, for example, optionally mounted on motorized gimbaled platforms providing addition aiming or alternative angles of irradiation of surfaces or objects (e.g., rotating a projector to scan, paint or otherwise illuminate the shadows of objects on a table or surface). In one embodiment, the angle of attack of an irradiation beam(s) may be varied. Such embodiments may irradiate viruses from multiple or repeated multiple angles to deliver a more effective irradiation to—e.g., overcome shadowing effects of irregular surfaces.

In one embodiment, light radiation is guided to MEMS devices or other projection or modulation apparatus(es) through free space or via fiber optic cable or bundles of fiber optic cables. In this manner, a number of different projection devices can be illuminated by a single light source. The various projection devices may be powered (illuminated) by the bundles at the same time or they may be multiplexed in time, for example.

A typical DMD device turns on/off a small portion of light in a beam reflected off the DMD. Other types of mirrored devices actually direct light reflected off mirrors of the device to a specified area. Both types of DMDs/reflectors may be utilized in the present application. Illumination from more than one DMD may be applied in series or parallel and be configured to either increase irradiation power at various points in the beam and/or reduce or eliminate irradiation power at other points, such as when scanning, illuminating or painting an object in either positive or negative space. Such embodiments of the application can be roughly described as putting an object in a protective bubble where all spaces around the object are irradiated and disinfected effectively placing the object or person in a bubble. Such irradiation might include floor spaces around or along a path of a patron, and may include irradiating clothes or certain clothes (such as shoes, shoe soles, and/or edges of shoe soles) of the patron.

In yet other embodiments, sources of disinfecting light may be directly illuminating an area (i.e., without the use of reflectors/DMDs etc.). In one embodiment, an array of UV light sources (e.g., UV LEDs) may be individually modulated from fully ON to fully OFF and intensities in-between. UV-friendly (i.e., transmissible) optics may control the spread of the UV illumination as desired to cover a particular area of an indoor venue. Several such direct illuminators may be employed to cover as much of the indoor space as desired. People/pets may move around the venue in real time and individual light sources may be modulated in order to avoid direct irradiation.

Such person-in-a-bubble embodiments may be extended to people in professional fields such as nurses, doctors, surgeons, pathologists, coroner's, morticians, etc., essentially any person in any field where bacterial or viral transmission is a risk may be enveloped by a protective bubble according to the present application.

As noted elsewhere the system may include a scanning mechanism, from expanded laser beams or LED light (irradiation) sources or wider beams from bulbs of diffused narrow sources, for example. In various configurations one source of light may not be sufficient to fully cover an area desired to be disinfected and multiple scans or multiple beams from a same or different light sources (or different MEMS devices, for example) may be employed.

Although it may be desired that mixing of beams is done simultaneously, or separated by a desired time difference (e.g., as in the normal case for adjacent scans from a same light source/device), the beams may be separated in time but the total amount of irradiation and virus/germ killing is maintained by the total amount of power irradiating the surfaces, and that total amount of power or number of irradiations may be altered to disinfect, even when the scans are separated by substantial time differences. In one embodiment, the scans are repeated often enough that there should be sufficient irradiation scans to effectively kill the target viruses and other undesirable substances. Desired duty cycles of disinfection by the one or more light sources may be scheduled to disinfect a desired amount (e.g., 60-90%) of the available airspace and/or surfaces within a desired period of time.

As noted elsewhere the direct light source and/or MEMS/projection device (collectively, “UV emitter”) may cast a desired footprint. The footprint may be electronically or mechanically varied. Such variation may be to more efficiently scan, illuminate or paint a surface or match an object to be irradiated. For example, when irradiating air, a wider footprint may be utilized to efficient cover air spaces close to the UV emitter and a narrower footprint may be utilized to provide more concentrated energy further from the UV emitter. Such air irradiation may be utilized to kill viruses in air above and around patrons, and may include use of footprints or beams of varying sizes and qualities.

In various embodiments, light beam intensity may be calculated, estimated and/or modeled to produce a desired energy density—measured in, e.g., joules/meter² (J/m²)—through air or on surfaces. In one embodiment, the intensity may be based on the determined amount of energy density needed to achieve a desired log reduction in pathogens. Depending on the power density (e.g., in Watts/meter), it is a straight forward calculation to determine the amount of time at such power density to achieve a desired energy density in a given airspace or on a given surface.

As noted above, the disinfecting light or irradiation may come from various sources. Artificial sources may include mercury vapor lamps, halogen lights, high-intensity discharge lamps, fluorescent and incandescent sources, and lasers. Such sources may include phosphors irradiated by other light wavelengths.

The present application includes the mounting of lights/lasers in/on a ceiling (e.g., corners, center, an array, honeycomb pattern, etc.), floors, backs of chair (e.g., illuminating chair behind) in similar or different patterns/arrays, above and below railings, near frequent touch spots, etc.

The present application may be embodied as a device, apparatus, mechanism, or other configuration having a UV light source and control mechanisms including for direction of light beams emitted from the light source and for directing control of those light beams via decisions of when to illuminate an area, space, or object with the UV light and how to direct the light beam through or to that area, space or object.

The present application includes methods of controlling qualities of a light beam which may include, for example, direction, speed, and timing of illumination. Such control may be automatic based on environment and population of a venue. Such control may include pre-designated areas of known high traffic or patron contact or areas of difficult to disinfect surfaces and such surfaces or areas may be provided a larger portion of available disinfection time/power. Such pre-designated areas may include items such as door handles, railings, armrests (or specific portions of such items such as the palm or finger end of an armrest receiving a majority of the disinfection time/power allocated to the arm rest or seat.

### Some Operating Environment Embodiments

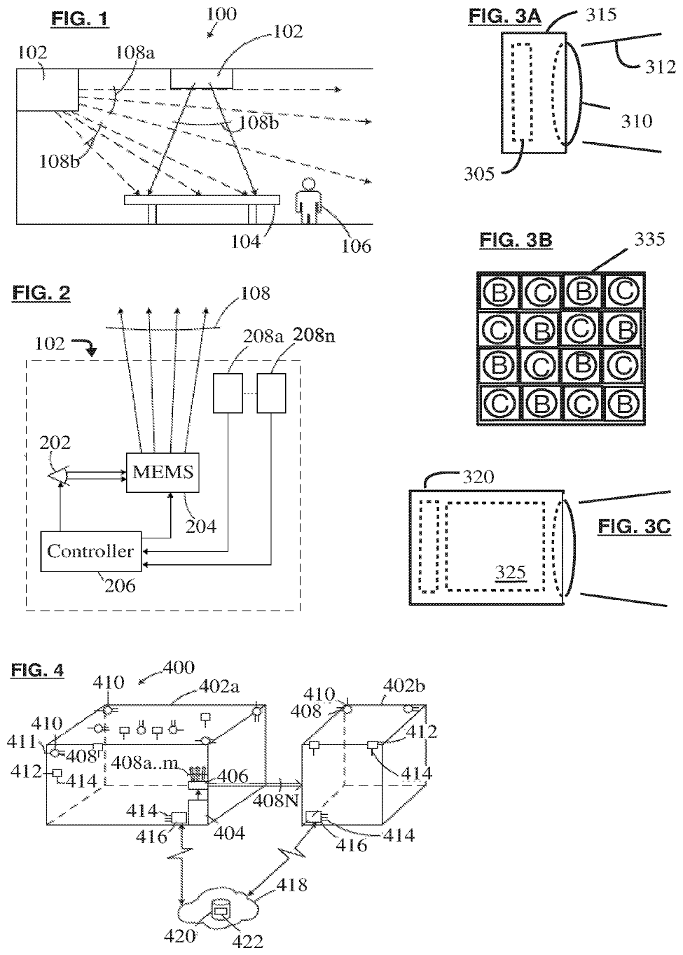

Referring now to the drawings, FIG. 1 is a side view drawing of a venue/space 100, such as a coffee shop, office, theater, medical office, medical room, operating room, forensics room or the like having air space, objects (e.g., surfaces, tables and countertops 104), and people (also, possibly animals) 106 with operating UV emitters 102 according to an embodiment of the present application.

The system 102 comprises a set of UV emitters, a set of sensors and a controller. As will be described further herein, the sensors may be placed in several locations within a venue. The sensors can detect all objects moving and/or stationary within the venue. In one embodiment, the sensors may detect living beings particularly and give location data to the controller. The controller may then send control signals to the UV emitters to direct light where and when desired to avoid directly irradiating living beings. In another embodiment, markers may be placed around the venue, showing where light may be directed safely—if there is a clear line of sight from the UV emitter(s) to the marker. In this embodiment, there may be no need to detect living beings directly in order to safely disinfect desired airspace and/or surfaces.

The sensors can detect objects in the venue, at least according to one characteristic. For example, image cameras/sensors can detect position and motion of objects in the venue, IR sensors can detect the heat signatures of objects in the venue, audio sensors can detect noise emanating from objects in the venue, UV photodetectors can detect UV light that may be reflected/scattered by (or emanating from) objects in the venue.

In reference to FIG. 1, UV emitters of system 102 are shown emitting directed light beams 108 to volumes of ambient air, surfaces, high contact areas throughout the venue, such as tables 104 and countertop 104. It may be seen that more than one UV emitter may occupy the space/venue and supply light 108 to the same and/or different surfaces at various times.

The placement of such systems may be determined by the sanitation and/or disinfecting needs of the venue. For merely one example, if venue 100 is a cafd, bar or restaurant, then UV emitters may be placed in the corners or other parts of the ceiling (or, alternatively floors, walls, etc.) of the venue—to provide, e.g., a crossfire fusillade of illumination on any given surface. Other venues/rooms may desire different placements of UV emitters throughout.

Continuing with this embodiment, if a table is vacant and the beams can inundate the table, as there are no persons/patrons. Countertop 104 is mostly vacant, but patron 106 is avoided by the system by identifying and locating the position of patron 106 and directing the irradiation so as to avoid patron 106 (as will be discussed further herein).

The table that has patron 106 near it may provide a challenging irradiation pattern. The full system (i.e., with multiple UV emitters) may, for example, identify lines of sight (for each possible UV emitter) to various points/areas on that table and irradiate if no persons are currently obstructing the line of sight for a given UV emitter. This line of sight illumination may dynamically change over time, depending, e.g., on the movements of people/pets. If that happens, the system detects those changes and may turn on/off the illumination accordingly. As discussed further herein, the full system further comprises a set of sensor that supply data to a controller. The controller sends signals to the UV emitters to dynamically control the illumination to safely irradiate a venue.

As may be seen further in reference to FIG. 1, system 102 may autonomously steer light beams 108a to miss patron 106. As may also be seen, countertop 104 may be being irradiated with irradiation subset 108b from two or more systems 102. Certain areas within the venue may always be free of people and therefore available to be painted on a continuous basis, nevertheless the system may ascertain a clear status of the irradiation paths prior to any energization. Irradiation subset 108a is shown only irradiating space above patron 106c's head. Identifying height, locations, motion and/or velocity of patrons may be used to determine a clear shot for irradiation. A clear line of sight and/or space between the irradiation path may be used for or as a validation of a clear shot. For these embodiments, the controller (e.g., a part of system 102) may be employed to control UV emitters in at least one characteristic of operations—e.g., ON/OFF, intensity, direction, pulse, or the like—in order to place UV light where and when the system deems it safe to employ UV light so as not to irradiate living beings in the venue.

### Reflective System Architecture Embodiments

FIG. 2 is a circuit diagram/architecture of one irradiation system of the reflective type, according to an embodiment of the present application. In one embodiment, system 102 may comprise controller 206, which comprises a set of data and control lines. For example, controller 206 may be connected to a light source 202, reflector 204, and one or more sensors 208a-208n.

As mentioned herein, light source 202 may comprise one or more of sources of disinfecting light—including incoherent light, coherent light and/or a combination of both incoherent and coherent light. In addition, in some embodiments, light source may further comprise one or more sources of visible light—e.g., to be combined with the disinfecting light (which tends to be invisible). In some embodiments, the use of visible light can give people an indication of where the disinfecting light is being applied—or a warning indication that the disinfecting light is not being correctly steered. In some embodiments, light source 202 may be controlled by signals from the controller—e.g., to turn on/off, modulate intensity, Pulse-Width Modulate (PMW) or otherwise control the energy output, the wavelengths emitted, the timing of light output or the like, as desired.

The light from light source 202 may be directed to one or more reflectors 204. Reflector 204 may be any of the known or unknown types of reflectors, as mentioned herein—including: DMD, MEMS, LCoS, gratings, etc, or the like. Reflector 204 may be controlled by signals of the controller 206 to steer the light to any desired location within the venue/space. Reflector 204 may be used to disperse light to as wide or narrow a beam as desired. For example, in some instances, a wider beam may be desired to disinfect a large surface area. In other instances, a narrow beam may be directed to a point of interest as desired. The reflectors may comprise a set of individually controllable mirrors. These mirrors may be made from polished aluminum (with or without any coatings applied to the surface) or any other metal having good reflective characteristics of UV light.

The controller is configured to receive information (e.g., any of positional data, echo location, image data) from one or more sensor(s) 208a-208n. These sensors give real-time data as to the location of desired surfaces and the location and velocity of persons within the venue/space. Such sensors may comprise: image/video cameras, stereo vision, LiDAR, radar, heat/IR sensors, ultrasonic sensors, UV photodetectors, audio microphones and the like.

This sensor data allows the controller to receive or calculate positions of patrons and/or determine clear lines of sight. The use of this sensor data to do the following: identify objects, track objects, hit objects with disinfecting light, and/or avoid objects (e.g., in the area of self-driving vehicles) are well-known in the art. For merely one example, such data and corresponding controller calculations are found in this reference in the art of autonomous self-driving cars: (1) US Patent Application 20200057443, DETECTING AND RESPONDING TO PROCESSIONS FOR AUTONOMOUS VEHICLES—which is herein incorporated by reference in its entirety. Other such references are well known.

System 102 may also employ machine learning to recognize relevant disinfecting surfaces and persons and their motions within a venue/space. One example of such processing may be found in the following reference: (1) US Patent Application 20190033085, NEURAL NETWORKS FOR VEHICLE TRAJECTORY PLANNING—which is herein incorporated by reference in its entirety. Other such references are well known. Such person and surface recognition APIs or software developed for self-driving cars may be used to identify people and their precise locations from which calculations and/or models to determine clear shots to be made.

In some embodiments, one or more sensors may be, for example, an audio device such as one or more microphones or include a microphone and inputs audio, audio signals, and/or results of audio analysis to the controller. As will be discussed further herein, sound data may be used to detect a sneeze and/or cough from a patient and its location (e.g., through echo location or the like) and used to steer a disinfecting beam into a cloud of pathogens therein. Such sound location calculations and determination may be found in: (1) US Patent Application 20170328983, SYSTEMS AND METHODS FOR TRANSIENT ACOUSTIC EVENT DETECTION, CLASSIFICATION, AND LOCALIZATION—which is herein incorporated by reference in its entirety. Other such references are well known.

It will be appreciated that any of the sensors may be placed and located either at the unit housing the reflector 204 and/or at any other places within the space/venue to optimize the quality of the data desired to be collected.

It will also be appreciated that the controller may be co-located with system 102 unit or, alternatively, may be located in any other place(s) within the space/venue. In other embodiments, the controller may be remote from the space/venue and may be hosted on a network/Internet and the control may be provided as a service.

It will also be appreciated that the light source(s) may be placed in one area within the venue/space and the light emitted from the system may be distributed to different emission ports located at different parts of the venue/space via a suitable light guide or through free space. This may be desirable if the cost considerations to disinfect the venue/space with multiple, distributed light sources throughout the venue/space is prohibitive.

### Direct UV Emitter Embodiments

FIGS. 3A through 3C show a number of embodiments comprising direct UV emitter sources. A light source 305 disposed to illuminate a lens 310 forming projection 312. The light source 305 may be, for example, a UV light source (that is controllable by signals from the controller for ON/OFF/modulation control) comprising one or more bands of UV wavelengths. In one embodiment, the light source comprises a plurality of light sources. In one embodiment, the light source comprises a UV light source or sources (e.g., a set and/or array of individually controllable LEDs or the like). In one embodiment, the light sources provide a series of substantially parallel rays of UV light (or one or more wavelengths, or one or more wavelength bands). The projection lens 310 may be of any form and may be constructed from UV friendly (e.g., UV transmissive) materials such as quartz or silica, for example. Such UV friendly lenses may be acquired from Fresnel Technologies Inc. (www.fresneltech.com) or can be made from UV friendly resin from Momentive Inc. (www.momentive.com)—e.g., their Ultra Clear LSR7XXX resin products.

The projection lens 310 may include anti-reflection coatings such as UV-A and/or UV-B anti-reflection coatings. The light source 305 and projection lens 310 may be enclosed in a housing 315 evacuated (vacuum) and/or filled with a UV friendly gas (e.g., nitrogen, pure noble gases such as argon, helium, krypton, neon, radon, xenon. A combination of gases may be utilized). In one embodiment, the UV housing may be filled with a UV friendly gel or transparent material (e.g., non-oxygenated gels). Projector 320 may include a light conditioner 325 which may act as a collimator, homogenizer, or other optical elements. The light conditioner may be constructed from, for example, UV friendly material (e.g., UV transparent or substantially transparent and/or does not degrade substantially in the presence of UV light, such as quartz, silica or any other known UV friendly material).

FIGS. 3A through 3C also illustrate various light source arrays according to embodiments, any of which may be utilized as, for example, light source 305 or in other embodiments described herein or in other projector designs not described herein.

Light source array 305 is in a 5 dice pattern comprising 4 corner sources and a center source. As noted elsewhere herein the sources may be a single wavelength, a wavelength band, a set of wavelength bands, etc. The sources may also be mixed mode, e.g., wideband and narrowband, coherent and non-coherent light in any mixture. In one embodiment, the light sources may be individually controlled on/off and/or regulated variable power (e.g., intensity) In one embodiment the four corner sources comprises a first emission and the center source comprises a second different emission. The center source may be, for example, a more intense source and/or more directed source designated for longer range.

A projection lens, if associated with the light source, may comprise a dual curvature such that a central portion of the lens may be curved to enhance or control the more intense, or more collimated, or otherwise different emission from the central source (and/or control a similar emission differently), and the remaining portions of the lens (e.g., outer portions of the lens) may be curved in a manner conducive to the type of projection desirable from the corner sources. The remaining portions of the lens may be ground in consideration of the curvatures of the central portion (or vice versa) and/or the corner light sources may be arranged such that they mostly (or entirely) illuminate outer portions of the lens. In one embodiment, the corner light sources may be replaced with a ring of light sources directly illuminating the outer area of the lens. In one embodiment, the corner sources emit UV-C wavelengths of light and the central source emits UV-B wavelengths of light. In one embodiment one of the sources may be a laser and the other source(s) may be wideband or narrowband UV.

The central portion may be configured (e.g. ground) to direct light toward more distant targets and the outer portions of the lens may be configured for near object illumination, for example. Similarly, embodiments with mirrors or other reflective or directive portions or elements may be configured for different emanations and/or projection spaces.

Light source 335 may be configured as a n×m array of UV sources (e.g., LEDs). As illustrated, UV-B and UV-C sources may be arranged in a quincunx pattern which, as with other described arrangements, is exemplary. Together, the sources may provide a relatively even source of well mixed UV-B and UV-C light.

Spread functions of adjacent LEDs (and/or next neighboring LEDs) may be arranged such that the lights mix together such that a full width at half maximum of adjacent (or neighbor adjacent sources) mix together in a range of greater than or equal to 0.25 d2 where d2 is a spacing of one source to its next neighbor source (or next source, or next source emitting same or similar wavelengths of light). The source may be configured for independent control of each source and/or energizing as a group or groups. Light source 365 provides a central array of sources 370 and corner sources and may be energized similarly as light source 305, for example.

### Set-Up and Training Embodiments

A flowchart/method embodiment is now disclosed for the set-up and training of the present system. Upon the initial installation of the present system, it may be desirable to set-up and train the system to assess the effectiveness and safety of the system in the chosen venue/space. Setup may commence by having a user/admin identify the various objects within the venue/space where it is desired to supply disinfecting light. Within a typical venue/space, there may be a number of objects for disinfecting—some fixed (e.g., installed countertops or the like) and some moveable (e.g., free standing tables, chairs, door knobs/handles).

In one embodiment, the system may be given a spatial map/model of the venue/space and within this special map, the system is given data correlating to both the location and any particular cleaning protocol associated with both fixed and moveable objects. For example, countertop locations would tend to be fixed and the contours of such fixed objects may be input into the controller memory and may be less subject to change during the operation of the system. In addition, any particular cleaning protocol associated with any object may also be stored in controller memory. For example, if the object is a countertop used for food preparation, then a more rigorous cleaning protocol may be desired than, e.g., a chair.

Models for the objects may be associated with various objects. For example, fixed objects may have the location and contours identified, as well as a cleaning protocol. For moveable objects, a model may be used to train the system to detect and/or identify the object over time. As these moveable objects may be found in different locations within the venue/space, the system may be trained with the models (e.g., via neural networks or other artificial intelligence models). As the system operates over time, the system may employ these models to identify moveable objects and apply any associated cleaning protocol. For one example, a table may have a more rigorous cleaning protocol than a chair.

These models may be applied by the system and, as a part of the system's installation, the system may be turned on to operate in a training session. For such training session, it may be desired to employ only visible light to test for beam steering and its safety. The performance of the system may be assessed and feedback may be applied to the system to correct/alter the models and the performance.

For certain examples, training of autonomous systems may be found in the following references: (1) U.S. Pat. No. 9,669,544, Vision-guided robots and methods of training them—which is herein incorporated by reference in its entirety.

As another part of the system initialization/set-up, another flowchart/method embodiment is given for training the system to operate with living objects moving through the venue/space. The process may start with user/admin supplying various models for the identification and detection of living objects that may be found in the venue/space. For some embodiments, the models for living objects may be associated with data from a heat sensor, as heat is a characteristic usually associated with living objects. Models may also associate the motion of a contour/outline of a size or shape that is typical for a human (e.g., from infant to adult) and other living objects (e.g., dogs or the like). The system may be initialized and started with only visible light to monitor the operation of the system. The performance of the system is assessed and feedback may be given to the system to correct its models and/or its performance.

In yet another embodiment, the system may employ a heat/temperature/IR sensor to detect a person who is running a fever before and/or at time of entry into a venue/space. A protective airspace may be affected by the system to surround that person (as described further herein) with UV light to protect other persons in the venue/space. An alert may be given to wait staff/personnel to inform them of the person having a fever and allow them time to ask them to leave and/or other instructions. The system may detect all of the surfaces touched by such persons and affect a disinfection of all of those touched/infected surfaces. In yet another embodiment, facial recognition technology could be employed to identify such persons and such information may be used by responding persons.

### Some Operational Cleaning Protocols

After the system has been installed and optionally trained through set-up, the system may apply a number of desired cleaning protocols. One process may start when there are no living objects in the venue/space. Such situations may be presented right before opening of a place of business or other desired spaces; after closing of business—or at any other time in which the venue/space is devoid of living objects.

In these cases, the system may employ its most rigorous cleaning protocol possible. In one embodiment, the disinfecting light may be in the UV-A, UV-B, UV-C, X-ray and/or microwave regions—e.g., radiation that may cause damage to living tissues, even if briefly exposed. To provide additional safety during these times, red light may be projected, together with such intense radiation, to give a visual indication to humans as to the location of such radiation.

When the venue/space is open to living objects (e.g., either by opening and closing times of the venue/space and/or the active identification of living objects), the system may opt to switch to less intense radiation that still has disinfecting affectation—e.g., UV-A, UV-B and/or U V-C. In those times, the system may add in visible light of one or more colors to indicate different operating modes. For example, a color perhaps different from red (e.g., yellow) may indicate some caution against exposure. If a patron sees a yellow (or red) light emitted from the system, that may be a clue for the patron to look away from the system, if such light is seen at all.

At other times, such as when an area has been disinfected, the system may emit only a green light to indicate that the area (e.g., table, countertop) has just been cleaned and that it is fine to use. Another use of visible light may be employ a different color—or to pulse colored light—to give an indication that the system desires to disinfect a given surface (e.g., after a patron has used or touched a surface and the system has detected such use or touching).

In other embodiments, the system may switch to stronger radiation in areas of the venue/space that are devoid of living objects and/or at times of reduced concentration of living objects.

The system may monitor the venue/space and receive data through its set of sensors to detect and/or identify the position and/or velocity of persons in the venue/space. The system may identify surfaces that are desirable to be cleaned, irradiated and/or disinfected. This identification may be accomplished via a set of criteria, comprising: whether persons have touched the surface, how long persons have been near the surface, how likely is it that a person exposed to the surface might be infected, etc.

Once surfaces are identified as ready for disinfecting, the system may make the determination as to whether any persons/living beings are in the line of sight of the disinfecting light beams. If yes, then the system could delay irradiation. If no, then the system could apply disinfecting radiation to the desired surface. If persons are in the line of sight of the light beams, then the system could use the colored light or pulsed colored light to give an indication to persons that that surface is to be disinfected—and move away, out of the line of sight. Alternatively, if the system desires to clean any area, the system may project another distinct colored visible light and/or flash/pulse any colored visible light at the area and/or its contour. If there is any obstruction (e.g., person, object or the like) that prevents the system from performing a disinfection (e.g., someone/something in the line of sight of the disinfecting light), then the system could generate some alert/warning—e.g., to wait staff, personnel, etc.—in order for some action to be taken to remove such obstacle.

It will be appreciated that, for the foregoing, any colored visible light for the various modes may suffice (including white light)—and that any flashing, illuminating, contouring and/or signaling protocol may also suffice to provide suitable information feedback to humans in the venue/space.

### Infectious Cloud Disinfecting Protocol

In many venues/spaces that are populated, it may be desirable to clean and/or disinfect the ambient air and/or surrounding surfaces after a person sneezes, coughs, expectorates, vomits or the like. A flowchart/method embodiment of a cleaning protocol to clean living infectious waste products is now described.

The system may monitor the venue/space that is populated by people for signs/data of infectious waste being deposited/spread by people. Such monitoring may be the collection of data from sensors placed advantageously around the venue/space. Such data may be one or more audio microphones/pickups that can detect sneezes, coughs, spit and/or vomit sounds (i.e., sounds associated with “pathogen cloud” events). In one embodiment, the system may employ a set of sound models that are associated with and can determine and/or model whether an infection event is a sneeze, cough, spit and/or vomit.

The system may apply these event models to locate and/or predict the spread of infection for such event. In some embodiments, the system may apply echo location from a set of audio microphones to determine the source of the sneeze, cough and/or any other event that has a sound pattern. In the example of a sneeze or cough or the like, the audio data can be used to determine which patron/person sneezed/coughed or the like. This may give a first order approximation/model of the area of infection needed to be disinfected. The audio data may be correlated with image data of the patron/person's head motion showing the direction of the sneeze, cough or the like. From such correlation, an infection cloud's contour and speed of spread may be modeled to provide a second order approximation/model of the area to be disinfected.

The system may apply disinfecting radiation to the infected area and/or the extent/contour of its modeled spread. The amount and extent of disinfecting radiation would desirably be sufficient to disinfect the entire contour of its infected area—and its potential spread. For one example, models of sneezes may be applied by using known data of the speed/velocity of infectious droplets that may be spread.

In yet another embodiment, the image/video sensors may be the first indication to the system that a person may be about to sneeze/cough/expectorate/vomit before any audio signals are picked up. In this embodiment, the system may make a best guess as to where the extent/contour of infection may spread and start to affect a disinfection routine. Audio signals that may arrive later in time than image signals may be used to refine and/or cancel the disinfecting protocol.

### Alternative Venue Embodiments

FIG. 4 depicts yet another embodiment of the present system. Neighborhood 400 may comprise one or more venues/spaces 402a and/or 402b that may be able to share resources of the system in order to reduce the cost of installation and set-up for one or more venues/spaces. As may be seen, a light source 404 may be located in one corner of a venue/space. As discussed above and elsewhere, such light source may comprise a set of coherent and/or incoherent disinfecting light sources—as well as a set of white and/or colored visible light sources for mixing with disinfecting light sources. Such light may be fed into an optical multiplexor 406 and light may be advantageously distributed to light ports 410 (as denoted by circular shapes) via a set of optical light guides 408a-m.

As previously mentioned, light ports 410 may comprise an input light guide—which can illuminate a DMD/MEMS reflector installed in the light port, to provide a scanning/painting ability of the light from the light port. As seen, these light ports may be distributed in corners of the venue/spaces, in the ceiling of the venue/space, or in other areas as desired.

A set of sensors 412 may be placed advantageously throughout the venue/space. Such sensors may be any of the previously discussed sensors (e.g., cameras, heat detectors, audio microphones, LiDAR, radar and others)—to provide data via communication lines 414. In addition, light ports 410 have communication lines 414 to provide control signals to the reflectors that comprise the light ports to steer the light as desired. Communication lines 414 may carry the data signals from the sensor and control signals to the ports to a communications port 416.

Communications port 416 may, in one embodiment, comprise a controller (with its own processor, memory and computer-readable instructions and cleaning protocols) for an individual venue/space. Alternatively, communications port 416 may be in communication to a network and/or computer 420 (that may itself be hosted in a network/cloud/Internet 418). The network may host the controller 422 that inputs sensor data from one or more venues/spaces and sends out control signals to light sources 404, multiplexor 406 and light ports 414—e.g., in order to affect a set of cleaning protocols and/or disinfecting routines to clean/disinfect each such venue/space.

It will be appreciated that the embodiment of FIG. 4 allows for the sharing of light sources and controllers in order to reduce capital costs of installation. In addition, the system may be run as a service from a network and/or the Internet.

### Controller Embodiments

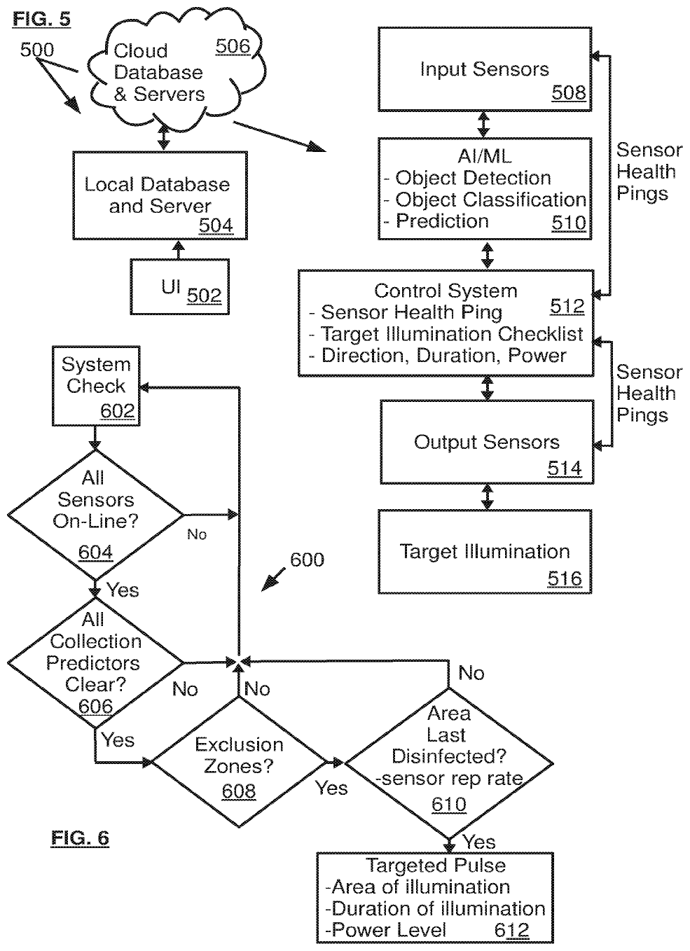

FIG. 5 depicts one architecture embodiment of the present controller system. System 500 may comprise an optional User Interface (UI) 502 through which users/operators of the system may communicate status and control states of the system and possibly access safety and operating data—e.g., from optional local databases/servers 504 that are in communication with control system 512 and/or optional cloud databases/servers 506. Apart from these optional components, the system may comprise input sensors 508 (as described above—image, video, photosensors, audio, etc.). The system may also comprise an Artificial Intelligent (AI)/Machine Learning (ML) block 510 that may detect objects, classify/identify objects (e.g., as living/non-living) and predict the motions of objects in order to schedule the UV emitters to irradiate/clean/disinfect a desired airspace and/or surface.

Control system/controller 512 may comprise a processor, computer readable memory comprising computer readable instructions that may execute the AI/ML block 510. In addition, control system 512 may control other aspects of the operation—such as sending out health pings to the sensors located in the venue, execute target illumination processes that may control aspects such as direction of light, duration of light and modulate the power and intensity of the light, according to many of the protocols described herein and in co-owned patent application as mentioned above.

In one embodiment, it may be desired to employ commercially available processors and routines—particularly in the AI area, such as NVidia. For merely some examples, it may be desirable to use Jetson Nano, Deepstream, Transfer Learning Toolkit, Jetson Inference Functions in the controller.

The system 500 may also comprise optional output sensors 514. These sensors 514 may be the same as input sensors 508—or they may be different sensors—to monitor the effectiveness of the light irradiation. For merely some examples, output sensors may be visible light cameras that detect the output of advantageously placed phosphors that convert UV illumination to visible light—and/or other markers (e.g., visible marks or active lights placed around the venue) that otherwise are detectable to determine clear lines-of-sight for UV irradiation. Alternatively, output sensors may be UV photodectectors that are measuring the amount of scattered/reflected UV illumination that is placed throughout the venue. Such UV photodetectors may be able to detect that—when a UV emitter is aiming its UV light at a part of the venue, the light does not reflect off of a metallic (e.g., aluminum) surface, thus creating strong ambient amounts of UV light in the venue. The system may then train itself to avoid illuminating that surface to reduce the amount of ambient UV illumination.

Lastly, the system may comprise target illumination/UV emitters 516 that would operate under control signals as to where to aim the UV illumination, how long to illuminate an area, and how strong the illumination is to be.

For another architectural embodiment of the control system, the system may comprise input sensors that take in input data from the venue. That data may be stored in an optional database to be processed by a predictive engine. The predictive engine module (perhaps stored and/or executed by the controller or in the cloud) may make prediction about whether an object is living or non-living, whether that object is moving or stationary, and if moving, where the object is likely to move. Controller may comprise a processor, computer readable memory comprising computer readable instructions. Controller may execute the relevant modules/routines for the operation of the system. Controller may also send out illumination signals to the UV sources in the venue, thereby directing a disinfection illumination/pulse. Output sensors (which may be the same as input sensors, or different sensors) may pick up signals that feedback to the system the effectiveness of the directed illumination.

For another embodiment, optional calibration and/or monitoring blocks of processing that may be stored and/or executed by the controller or in the cloud. Calibration module may be a routine that checks to see that every sensor is correctly calibrated. Such modules may proceed by having the system check to see if the individual sensors/emitters are operating properly—either passively (e.g., by checking whether the current data from a sensor is accurately correlated with previous sensor data from a properly operating sensor) or actively (e.g., by impulse testing the venue with illumination in a desired band (e.g., visible, colored light, UV, IR etc.) and checking the response). In addition to individual sensor calibration, the controller may check to see if the network (in whole or part) is properly calibrated (e.g., by pinging each sensor and check its health).

As the system is running, a monitoring block may periodically engage in network monitoring by pinging the sensors/emitters for health status checks. In addition, monitoring block may check the health of individual sensors/emitters during operation.

Once the optional calibration/monitoring blocks indicate that the system is functioning properly, the system may engage in normal operational modes. FIG. 6 is one embodiment of an operating module 600 that provides a desired level of safety during system operation. Module 600 may proceed with a system check 602 that goes through a number of optional safety checks prior to commanding an irradiation at 612. For example, system check could determine whether all (or a desired subset) of sensors are online and functioning at 604. System check could determine whether the system detects and/or predicts a clear line of sight for a beam of illumination to desired targets and/or areas. In addition, the system check could determine whether the area under consideration for illumination is in an “exclusion zone” at 608. By “exclusion zone”, it is meant whether the area contains materials that should not be illuminated with UV or other radiation—or it could be an area with a reflection profile that is not desired (e.g., perhaps metal creating specular reflection of light that places the ambient UV radiation above a given threshold). System check could also keep track of the frequency of disinfection for a given area at 610. The system may have a set of rules and/or heuristics as to how often and how much an area is to be irradiated and the system check could determine that it is still too early to irradiate. There may be a desired amount of time the system allows between disinfections of potential targets. If all of the desired checks and/or conditions are passed, then the system could command a disinfecting illumination of the airspace and/or surface in question at 612.

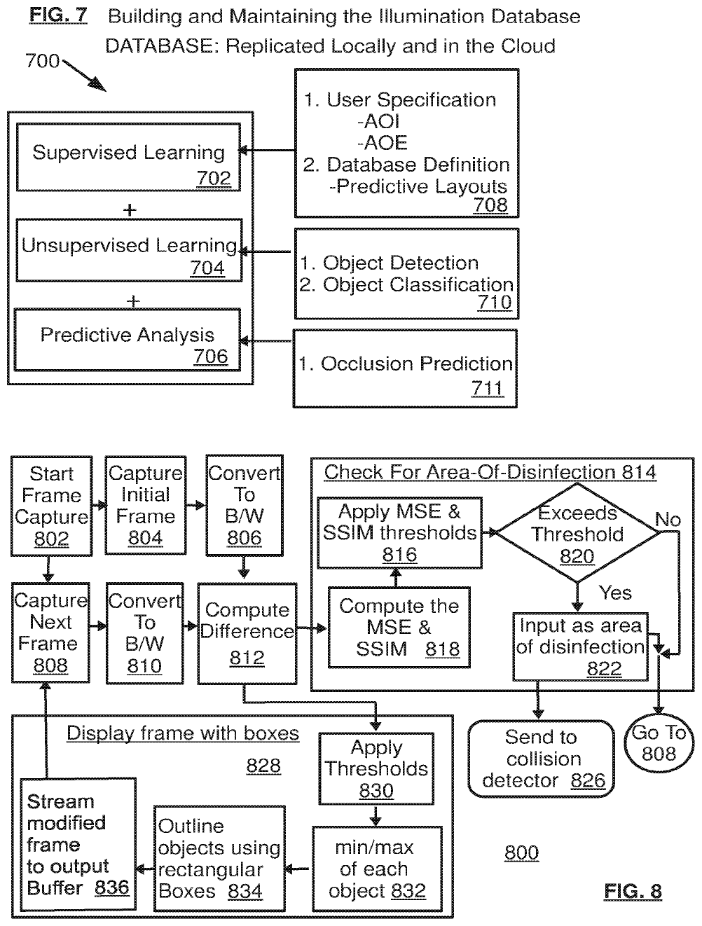

FIG. 7 is one embodiment of the optional database that may work in conjunction with other modules of the present system. Database 700 may comprise data servicing a number of routines/modules of the present control system. For example, database 700 could comprise storage and routines that support: (1) supervised learning 702 (2) unsupervised learning 704 and/or (3) predictive analysis 706—possibly amongst others. Supervised learning module 702 may comprise data and processes that help with the initial setup of the control system and embed some intelligence in the system from the outset (and even during operation). Such data/routines could comprise: Areas of Inclusion (AOI), Areas of Exclusion (AOE) and predefined layouts of the space/venue to populate storage and routines at 708. AOIs might comprise identifying areas that are high touch surfaces in the venue that may be cleaned often according to a duty cycle of cleaning. Another AOI might be predefined areas in which UV absorptive material has been placed in the venue—e.g., to allow regular irradiation cycles that, once completed, afford a desired percentage (e.g., 60-90%) of the airspace to be disinfected. AOEs might comprise areas to be avoided for irradiation—for example, metallic surfaces that could be areas of high UV reflectivity (and thus, increasing the ambient background UV illumination). Other AOEs may be areas that are identified as being made up of material that may react to UV or other irradiation. For merely one example, areas that are constructed of plastic may be designated as an AOE. Predefined layouts may comprise pre-identified objects, e.g., tables, countertops and other fixed areas in the venue, that may have some preference in terms of the need of illuminate (or not) and how frequent and with what intensity. For merely one example, a given UV emitter may be assigned a particular area within a venue to illuminate and not others. Restricting the UV emitter's area of operation in the near field of view for the emitter may lead to higher levels of accurate light steering. For merely one example, it may be desirable to limit the irradiation area of the emitter to operate in a given amount of floor space (or airspace, as the case may be)—e.g., 100-1500 square feet, depending, among other things, on the spread of the illumination of the emitter. If the UV emitter is allowed to illuminate in the far field of view, objects to be identified and avoided may become smaller—and the system may occasionally irradiate small living objects. This may be more of a problem if the system operates on various 2-D views of the venue—and less of a problem if the sensor data is integrated together to form a 3-D model of the space with which the controller can operate.

Unsupervised learning module 704 may comprise data and processes that operate and/or update during the course of system operation. For some exemplary embodiments, unsupervised learning may occur with the operation of an object detection module and/or an object classification module at 710. Overall, object detection may be employed to detect relevant objects in the venue/space. In particular, object detection may, in one embodiment, detect the movement and/or motion of objects in the scene. Objects that are moving are typically relevant for the system to take notice. Object classification module may take as input those objects detected by that module—and seek to classify them in a relevant manner. In one embodiment, object classification may discern the objects as either living or non-living. As part of usual operation, the system will not seek to irradiate objects that are classified as “living”. Some exceptions to this rule are possible. For merely one example, if the system detects that a person has sneezed (either through a video and/or audio feed), then the system may decide to irradiate that person temporarily with UV (e.g., UV-A, UV-B and/or UV-C—in combination or separately) in order to disinfect the sneeze cloud emanating from that person. Such temporary irradiation should be designed for maximal disinfection and minimal harm to the person. For example, the system may decide to irradiate a person with one level of energy in the UV-A, UV-B bands and perhaps a higher level of energy in the UV-C band (as it is generally understood that UV-C may be less harmful than UV-A and/or UV-B).

Predictive analysis module 706 may comprise an occlusion prediction process—such that as the system is operating and disinfecting a venue/space (e.g., perhaps according to a duty cycle of disinfection), the system may note the motion of living objects in the venue/space and predict whether that living object is likely to be in the way of a clear line of sight for its beams of UV light at 711. If the system predicts occlusion, then the system may decide to alter its normal duty cycle. For example, the system may turn OFF, modulate the intensity down when occluded, or switch to UV-C only when illuminating that area that is likely to be occluded.

Continuous change detection and optical flow are image process techniques for computing the change over time of a pixel (or group of pixels) that may be employed in this application. For merely one example, (1) United States Patent Application 20200072619 to FUKUI, published Mar. 5, 2020, entitled “MAP MANAGEMENT DEVICE AND AUTONOMOUS MOBILE BODY CONTROL DEVICE”—is herein incorporated by reference in its entirety. In one embodiment, gradient and acceleration (of change) may be used for predicting the trends in the pixel data. The gradient and acceleration may be used to predict (or forecast) the future position of an object (i.e., person) that went behind or under some sort of obstruction. The algorithm for the gradient operator may be based on a delta in pixel values across several images and the acceleration is the gradient of the gradient. Such processing may be useful in many different embodiments,—for example, to compute or predict a clear line of sight for shining UV light (i.e., collision avoidance).

### Operational Disinfection Embodiments

The present application may apply techniques of object detection, object classification and collision avoidance, as has been developed for the self-driving automobile industry. There are, however, some differences that should be noted. First, for a self-driving car, its “scene” changes dramatically every few seconds—as the car itself moves, along with other cars, bikes, pedestrians, traffic control signals and the like. For the application of disinfecting a venue, a sensor's location is not likely to change and so any changes to its view of the venue is largely due to the movement of animate and inanimate objects (which require different treatment for UV illumination). If the sensor's position does in fact change, then suitable image transformations may be applied to work to direct UV illumination based on the new position of the sensor.

FIG. 8 is one embodiment of a method/algorithm/module for processing a stream of data from a sensor to detect objects in a venue. While this particular figure is designed as processing a set of images captured by a camera/video feed, it should be appreciated that this method may be adapted accordingly to the data feed coming from any type of sensor (e.g., IR imaging, UV imaging, visible light imaging and the like). It may suffice that the sensor delivers its data in somewhat discrete units that can be compared with over a time period. In some embodiments, the data feed may be substantially continuous and the data feed may be manipulated in such a manner as to compare data from one time period with data from another time period—e.g., to capture the notion of objects moving in a venue.

Process 800 starts by capturing an initial image frame (or initial data feed) at 802. In one embodiment, the sensor may be camera that is capturing image data that is in substantial alignment with the UV emitter—so that what the sensor/camera “sees” is substantially the same view that the UV emitter sends out UV light in substantially the same view. It should be appreciated that if the sensor is not in the same place/orientation as the UV emitter, then an appropriate transform of the scene can be accomplished by the controller—so that the system is accurately directing UV light in the venue and not irradiating forbidden objects (e.g., people, pets, other living objects, AOE, etc.).

The captured frame of data at 804 may be optionally converted from color image data to black and white image data at 806. This step is optional and may be desirable depending on the limits of speed of computation of the system. If speed is not a limitation for substantially real-time processing, then the system may utilize all of the chrominance data in a full color image. On initial processing, the same first image frame is picked up at 808, optionally converted to B/W at 810—and then the difference between the two image frames are compared at 812. The image frames may be partitioned into blocks—and the comparison of two image frames may occur on a block-by-block basis. In the first instance, there is no difference—as it is the same image frame. The initial frame is retained for a desired period of processing to compare with future image frames for computing the difference at 812. The first captured image frame may become the “baseline” frame from which future/subsequent frames are computed for the difference. It will be appreciated that the first such frame may remain the baseline frame—or the baseline frame may be replaced periodically (or on any other basis) with a given future frame and differences would then proceed off of the new baseline frame. One reason why it may be desirable to update the baseline image frame is because inanimate objects may be moved over time in the venue—and if the baseline image frame never changes, then the newly moved object may be continuously evaluated as having “just” been moved. In such a case, the moved object may not receive a dose of UV illumination when a dose might be desired. In another embodiment, an AI/ML module may be able to remove the moved inanimate object from being flagged as having been moved.

For one embodiment of image processing, the image frame may be partitioned into a set of “blocks” and the difference calculation may be made block-by-block. As mentioned, the difference could be any suitable metric—e.g., change in luminance, change in edge positions, change in chrominance (if the system is using full color), or any combination of such metrics. The difference computation may be input into an optional module that places bounding boxes around objects (828) and a module that checks for areas of disinfection (814). At 830, the system may apply any desired thresholds of change inside any block (or other areas of comparison) to flag whether something has changed/moved within that block/area. If one block has changed, then it is possible that there is an object that moved—and has also caused changes over a threshold in adjacent blocks. In such a case, the controller may instruct any UV emitters sending light towards that block to turn OFF UV illumination (or otherwise modulate the light to avoid injury to living beings). A timer may be set to a desired interval in which UV light is kept OFF (or modulated). After the expiration of the desired interval, the system may decide to update the baseline image to a new image—and continue to process with the new baseline image. This may be desirable if the object causing the threshold difference in the block in question was something inanimate that got moved (e.g., a chair was moved, a trashcan was moved, etc.)—in which case, it may be desired that the system proceed with a new, updated baseline image. This decision may be overruled if other safety conditions are present—such as, the AI block determines that the object in the block is likely a living being (e.g., within a threshold probability), or if an IR sensor detects that the object has a heat signature that is consistent with a living being. In these cases, a new updated baseline may not be desired—and/or the system flags those blocks as Areas of Exclusion (and not to be UV irradiated).

At 832, the min/max dimensions (perhaps by neighboring blocks) is determined for that object. A bounding box (or other suitable shape) may outline the object in question at 834. The modified frame may then be streamed to an output buffer at 836 and used by the next frame capture at 808 going forward. The bounding boxes around objects may be desired—or it may be optionally not applicable and the process may proceed just on a block-by-block basis.

The computed differences between frames at 812 may also be input into the module that checks for areas of disinfection at 814. At 818, the Mean Square Error (MSE) and/or Similarity Score (SSIM) may be computed.

For merely one embodiment, one equation for the MSE (Mean Squared Error) could be computed as follows:

$$MSE = \frac{1}{m * n} {\sum }_{i = 0}^{n - 1} {\sum }_{j = 0}^{n - 1} {[ I ( i , j ) - K ( i , j ) ]}^{2}$$

Where:
m=Number of x-pixels
n=Number of y-pixels
I=Image 1
K=Image 2

For merely one embodiment, one equation for SSIM (Structural Similarity Index Measure) could be computed as follows:

$$SSIM ( x , y ) = \frac{( ( 2 {μ}_{x} {μ}_{y} + {c}_{1} ) ( 2 {σ}_{xy} + {c}_{2} ) )}{( {μ}_{x}^{2} + {μ}_{y}^{2} + {c}_{1} ) ( {σ}_{x}^{2} + {σ}_{y}^{2} + {c}_{2} )}$$

Where:

μ and σ are the statistical mean and standard deviation computed form the pixel values for an image x and y.

It should be appreciated that other metrics may be applied to determine the desired and/or threshold amount of change in an setting over a number of images are sufficient to note that a change amounts to a AOE (e.g., such as a person, pet or other living objects) should be placed in a bounding box and possibly being excluded from UV irradiation. In one embodiment, these metrics may be computer on a block-by-block basis over two image frames (or sensor data capture, in case of other sensor data being relevant such as IR, UV, etc.). In another embodiment, such metrics may be calculated over a different subsets of an image/sensor frame (e.g., multiple blocks instead of block-by-block). In yet another embodiment, the metrics may be tracked over a desired set of image/sensor frames to detect that if, e.g., one block has been detected as over a desired threshold between two frames, and no change is detected over multiple frames thereafter, then it may be the case that the change is due to an inanimate object was moved into/out from that block. In such a case, that block may be place on the AOI list for disinfection.

In these and other embodiments, AI/ML modules may optionally be consulted to discern/predict whether the objects moving/remaining stationary are likely to be “person”, “pet”, “table” or some other object that the AI/ML module may discern/predict. In these and other embodiments, other sensors (e.g., IR, UV, audio) may also be optionally used to discern/predict whether the objects moving/remaining stationary are likely to be “person”, “pet”, “table” or some other object. For merely one example, IR sensors that correlate in space and time with image/other sensor data that the block in question has an object that is warmer than the ambient venue temperature (thus, possibly indicating that the object is living). These confirmations may be used to optionally supplement the original image/sensor data to determine whether the block is on the AOI or the AOE list.

### One Exemplary Embodiment

For merely one example suppose, once calculated, thresholds may be applied to the MSE and SSIM measures at 816 and if the thresholds are exceeded at 820, then the area may be designated as a “Area of Disinfection” (AOD) at 822—that is, an area (block or blocks) that are eligible for UV illumination. The AODs may then be sent to the collision detector module at 826. The collision detector module 826 will determine whether there is a clear line-of-sight for directing UV light to that AOD. If there is an animate object that appears to be moving into the AOD, then the system may suspend its UV illumination or otherwise modulate the UV to a lesser intensity or switch bands (e.g., from UV-A.UV-B to UV-C). Thereafter, a next frame is captured at 824 and sent to the beginning for processing.

Other alternative embodiments are possible and the scope of the present application encompasses them. For merely some other embodiments, it may suffice that the sensor data of a venue notes changes in the field of view. Those changes may be correlated to objects that are moving. Moving objects may be classified as either living or non-living. To determine the classification, many embodiments are possible. For one example, another sensor's data may be available to correlate with a first sensor to increase the likelihood that an object is living. To continue with the example, an image/video camera may note motion of an object in its field of view. The temperature of that object can be discerned by FLIR or some other IR photodetector. If the objects temperature is above the venue's ambient temperature, then it becomes more likely that the object is living (and more so, the closer the temperature gets to being an actual human's, dog's, etc. temperature).

In yet another embodiment, for the object that is moving, that image data may be fed into an AI/ML module for it to assess how likely the object is living. If the probability is over a desired threshold of living, then the system can tag that object as living and place it on a list of excluded objects for UV illumination. Some exceptions may apply—e.g., if that person sneezes and/or coughs, it may be desirable for the system to illuminate them for a small period of time (smaller period for UV-A, UV-B and perhaps longer illumination times for UV-C). The AI/ML module may be built in any suitable manner known in the art—e.g., rule-based, heuristic-based, neural network or the like. In some embodiment, it might suffice to employ the AI/ML module alone, if it is sufficient to identify living objects amongst a group of objects and can place a bounding box surrounding those living objects and treat the bounding boxes as AOE/zones of exclusion.

### Safety Check Embodiments

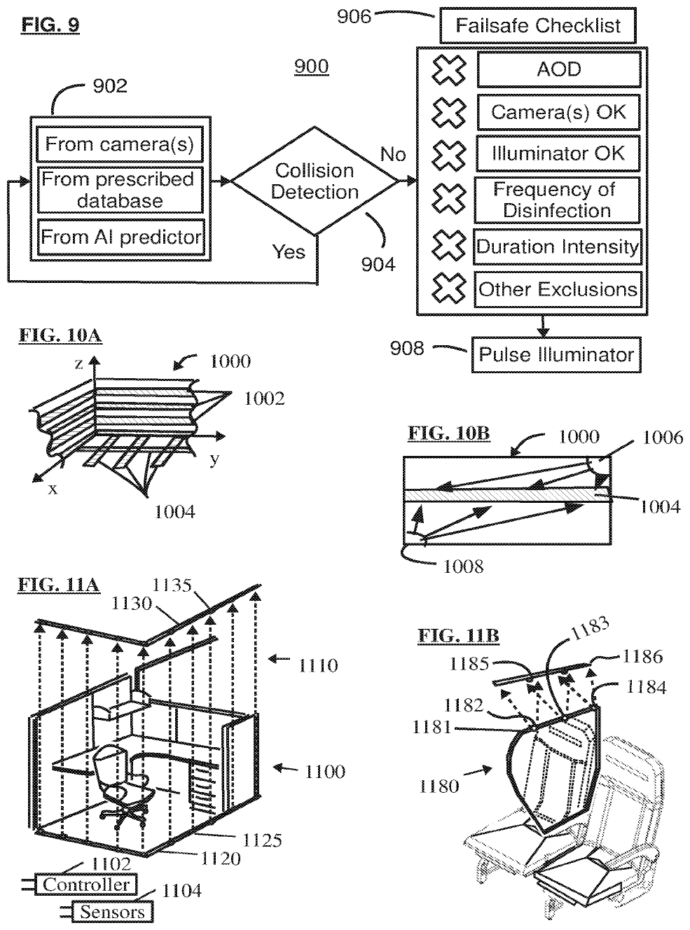

FIG. 9 is an embodiment of a safety check process made in accordance with the principles of the present application. Process 900 may begin by have the collision detection module/process input relevant data 902 from various sources to check if it is safe to consider commanding UV illumination. Data sources 902 may comprise one or more of the following: data from sensors (e.g., cameras, IR sensors, etc.), data from the optional database (that may comprise data as described above—AOE, AOI etc.), and data from the AI/motion predictor (that may predict whether there is an object moving into the line of sight of the UV light). If the data checks out that it is not yet safe to consider commanding a UV illumination, process 900 may cycle iteratively (or otherwise) through the data—unless and until the situation becomes clear. If the data indicates that it may be ok to illuminate (and hence, no discernable collision detected), then the process may consider a failsafe set of conditions 908 before there is an actual command to irradiate with UV. Such failsafe set of conditions may comprise one or more of the following: is the area and area of disinfection, are sensors working, is UV emitter working, does this illumination fit into the duty cycle of disinfection (that is, is this within the desired frequency of disinfection for the area), what duration and intensity to use, and any other exclusions applying here. If the failsafe checks are ok, then the process can command a UV illumination at 908. Otherwise, the process may iterate through the data and the checks unless and until all conditions are satisfied for UV disinfection. It should be appreciated that the determination on the failsafe conditions may be affected in several different manners—e.g., a desired threshold test, heuristically, empirically, probabilistically, AI, ML or any other suitable and known method for deciding.

### Location, Duration and Intensity Considerations

As for any decision on the duration and intensity of UV illumination, the controller may take into consideration that actual venue and the application needed to disinfect. For example, in one venue (e.g., a café, restaurant, warehouse, etc.), it may be more desirable to disinfect the airspace of the venue to tamp down the viral load of an airborne viral pathogen. In yet another venue (e.g., meat/food processing, hospital, etc.), it may be desirable to disinfect meat or other surfaces of bacterial pathogens that may be in water on the surface in question and have surface irregularities that may shield the pathogens from direct UV light. In both of those examples above, there is known in the microbiology literature how much light (measured in, e.g., Joules per square meter) is needed in order to obtain 1-log reduction (90% kill), 2-log (99%), etc. For example, it is known in the literature that it takes approximately 10 J/m2 to achieve 1-log reduction for an airborne pathogen like coronavirus—while, in order to kill salmonella in water, may take 30-300 J/m2.

In one embodiment, the system would know what the relevant pathogens are in the venue and their environment (airborne vs. water or the like) that are desired to be reduced. From that data, the system may have stored in its database the particular energy density needs to achieve whatever level of pathogen reduction is desired. From that data, the system can schedule a duty cycle on the venue/environment for UV disinfection.

For merely one example, suppose the venue is an indoor space like a restaurant, café, warehouse and the system goal is to have 1-log reduction of a known pathogen that takes 10 J/m2. In that case, the system may have a model of the available airspace and surfaces to disinfect. From this model, the controller may schedule a sweep of UV light through a desired level of venue airspace (say 60-90% of the available airspace). As the controller may have as data the power (in watts) of the UV source and a model of the spread of its light in the venue, then the controller can calculate (or it could be pre-calculated and stored in the database), the amount of time at a given level of power/intensity to spend sweeping that airspace to achieve that goal.

For another example, suppose the venue is a meat processing facility and the goal is to kill salmonella that is found on the wet surface of a meat carcass to a desired level of reduction, then the controller can schedule the time and intensity on the meat carcass to achieve that level of reduction.

For merely one embodiment, the controller could proceed as follows:
1) determine the pathogen and its environment desired to be reduced;
2) determine the available power/intensity of the UV source(s) that could irradiate a given area of airspace and/or surfaces to be disinfected;
3) determine the time and intensity in a given area to illuminate with each UV source to get to the desired amount of energy density;
4) if there is need for multiple passes of UV illumination to achieve the disinfecting goal, keep track of the previous amounts of UV light placed in that area and continue until the desired amount of UV light has been used.

Tis last process step may be desirable if the system detects pending collisions and needs to stop illumination (however brief) and resume when possible. In other situations, it may be desirable to sweep an area with UV light (rather that to train the UV light on a given area, airspace and/or surface) until the desired amount of UV light has been applied and continue on to the next target. As the effects of the UV light may be cumulative, it may be desirable to apply a portion of desired UV light in a first area, then move to a second area—and come back for the balance of the UV light on the first area after a period of time.

Such processing of the present system may determine a number of possible duty cycles that the system may perform to irradiate a desired amount of airspace and surfaces. These duty cycles may be schedules together or separately (e.g., airspace duty cycles and surface duty cycles scheduled together or separately). In addition, these duty cycles may be subject to change depending on a number of factors/conditions. For example, suppose an airspace duty cycle is operating to disinfect a desired amount of available airspace (say 60-90%). It may be the case that, in order to complete the duty cycle, the system would like to target specific locations in the venue to complete it. If a living person moves into an area needed to complete that particular duty cycle, then the duty cycle may skip over that area and move onto another area—or it may be interrupted to perform another duty cycle (such as surface disinfection) unless and/or until the area is clear of the living person. In another embodiment, the target area may be changed to avoid irradiating living beings. For merely one example, one target area may be on the floor of the venue (e.g., to clean that intervening airspace). If a person walks into that area, it may be the case that a UV emitter may aim slightly to the side or over the head of that person to approximately clean the desired airspace. The system may store which areas where completed according to the duty cycle and which areas were not completed. For those areas not completed, the system may store that data and monitor for a time in which the area becomes clear and then perform the irradiation.

### Safety-In-Layers Embodiments

While several cleaning protocols/methods have been described herein, there are a number of combinations of safety protocols that may be employed to further ensure that no living beings are being irradiated.

As described above, the system may start analyzing a venue via performing differential image frame-by-frame analysis. Each image frame may be partitioned into blocks (either non-overlapping or overlapping) and each block may be tracked over time. If there is some relevant change (as described above), then it is likely that something is moving within the region of that block. In one embodiment, the fact that something is moving in that block could set a presumption that that something is living—and the system is commanded to NOT send UV light into that block. Taking one exemplary case here helps to describe the method/process. Suppose a person is walking into a café (or the like) and sits down quietly for a period of time (e.g., 1-30 minutes, enough time for the system to detect no movement in the blocks that the person resides in). In that case, it is still desired that the person (who is still) continue to be NOT illuminated by UV light.

Towards that end, the following is one suitable process embodiment:
(1) for blocks that indicate change (e.g., motion), ensure no UV light is sent to those blocks;
(2) set a timer to a desired period of time in which no UV light is sent into those blocks (and subsequently turned by ON) until additional safety checks are made—the timer may be reset if motion is continuing to be detected in those blocks;
(3) if there is an optional AI module that can detect whether an object is a living being, and that AI module determines that the object in those blocks is probably a living being (to within a desired probability), then continue to not send UV light into those blocks;
(4) even if the AI module does not determine/predict that the object is living, if there is an optional IR sensor that detects a warm/hot body is in those blocks (e.g., a heat signature consistent with a living being), continue to not send UV light into those blocks.

The effect of the above-processing should be that a person who enters a space, sits down quietly and without motion, should be safe from being irradiated with UV light. In addition, on the step where an IR sensor may detect a hot head of a person (but that the body may not register too much heat above ambient) then the system could be trained to assume that the hot spot is the head of a person—and model/predict where the “body” of the person would be relative to that head; and that the system should not send UV light to areas/blocks “below” the head, where a body would likely be.

### Some Example Venues

The present system should be robust enough to serve several possible venues/spaces as processed by one or more of the embodiments of the present application. For one example, consider a subway platform in which people are waiting/walking around a platform—some boarding a train, some departing a train, etc. The embodiments of the present application may process this by image frame(s)—i.e., people (and other AOE) are detected and are not potentially subject to UV irradiation and other areas are detected and designated as AOI. It should be appreciated that that, while the AOI are subject to UV irradiation, it may be the case that the system projects UV light into the entire AOI. It may also be the case that subsets of the AOI will be subjected to UV irradiation, according to some desired duty cycle. For example, if the duty cycle is to target some subset of the available airspace/surfaces, then some proper subset may be scheduled to be disinfected by UV light. If the population density of persons in too great, the system may default to performing upper room/lower room disinfection, so as to avoid irradiating persons. Of course, if it detected that someone sneezes in such a dense population (as may be detected visual by a jerk of the head consistent with a sneeze and/or heard by an audio pickup and possibly triangulated to the sneeze's position), it may be the case that the system may temporarily irradiate persons for a small period of time to disinfect the cloud of pathogens. In such a case, the system may use UV-A and UV-B for a first small period of time and UV-C for a longer period of time (as UV-C is thought to be less harmful than UV-A and/or UV-B).

Another venue may be a hospital room where doctors and patients are placed in an AOE and the possibly rest of the room is placed in a AOI. The system may be able to place UV light between individual persons in the room—in order to cut down on the possible transmission of a Hospital Acquired Infection (HAI).

An indoor shop may be another potential venue (such as, a collectable shop—that may have—e.g., a ceiling fan that is constantly rotating). A patron can be seen moving around the shop and is placed in an AOE. In this case, it should be noted that, while the ceiling fan is moving (and hence may be processed as an object that might be living or not), the system has made the decision that the fan is not living and therefore is a candidate for UV disinfection. As discussed herein, that decision may have been aided by confirmation via a IR sensor (and seeing that its temperature is more consistent with being at room temperature than with a living being); or the system may have consulted with a AI/ML module to predict or otherwise determine that the object is not “human”, “pet” or other living object (or perhaps predict/determine that the object is a “ceiling fan”); or the fan was a “known” object in the environment and was pre-selected as an object on the AOI list prior to the operation of the system.

Other various embodiments of how the system might discern objects of interest (either as AOI or AOE objects) are considered. For example, a scene may be a room in which a dog is moving around. In this case, the system may use an image/video camera and the delta block-by-block processing as described herein to detect the dog. In that scene, the system detects both a person and an inanimate stuffed animal with the use of an image/video camera and object classification/identification from an AI/ML processing module (that may be hosted on a N VIDIA Jetson®—or other suitable hardware/firmware/software capable of doing so). In some cases, the system may discern a person in a room with the use of an image/video camera and an IR sensor to cross-reference.

### Hotspot Detection

An embodiment of a process for detecting areas of high traffic for both airspaces and surfaces is now described.

For merely one example, consider a venue (e.g., a store) in which people walk around in real time and may touch surfaces, objects, etc. As may be seen, a patron is walking through the store and is detected by the delta processing (e.g., on an image block by block basis or the like). The system also notes that a fan is moving nearly continuously. The system over time may compile statistics and/or other measures as to areas in the image where there is high movement/high traffic volume (i.e., “hotspots”) detected. In one embodiment, the statistics used may be maintained cumulatively over time as a histogram or the like. A colorized map (which will be cross-hatched in the black and white version of the picture) shows the outcome of such cumulative statistics—the fan's heat map as and the store's corridor (where most people walk) are depicted as the “hotspots” in this venue. The system may use these hotspot detections to move up a priority queue for disinfecting the venue.

As merely one embodiment of this process, the following is given:
1) create a histogram (or other suitable measure, statistical or otherwise) of delta changes (showing movement or another suitable metric) in the area of one or more sensor data (e.g., block by block in an image/video camera feed);
2) note which areas have the highest changes over a desired period of time;
3) optionally determine whether the delta changes are due to animate objects moving in those areas—if it is determined that the delta changes are due to inanimate objects primarily, those areas may be ignored or otherwise downgraded;
4) schedule a disinfection of the hotspot/high traffic areas/surfaces;
5) if desired, move those areas/surfaces higher up on any priority queue for disinfection if one exists.

### An Alternative Protocol/Method

As an alternative method/protocol for controlling the illumination levels of the various UV emitters in a given venue, the following is one that may not need to employ AI/ML techniques in order to avoid irradiating living beings in a given venue.

In one embodiment, a set of UV emitters, as described herein, is placed around a given venue with a set of sensors to detect clear lines-of-sight for directing UV light. A set of markers, lights and/or reflectors (hereinafter “markers”, collectively—e.g., anything that may be distinguishable by the system as denoted a target for illumination) may be placed in the venue, such that the markers may be detected by the various sensors in the venue. If the sensors can detect the markers (and hence are not obstructed), then UV emitters have a clear line-of-sight to illuminate the area around the markers, such that UV will not directly illuminate any object (that is, not only living beings, but any object that may be in the way of viewing the marker).

If there are sufficient markers placed in a row (or in some grouping), then the system may add an additional safety layer by only irradiating an area in which more than one marker is visible from a sensor whose view correlates to a safe application of UV light, if a desired number of markers are currently visible.

In another embodiment, the markers may be embedded in a UV absorptive material, such that the UV light directed toward the markers will traverse the venue (and disinfect the intervening airspace) and thereafter be absorbed in the material—thus, reducing the amount of UV scatter radiation back into the venue. In another embodiment, the markers may be embedded into a layer comprising retroreflectors. In such a case, the UV light hitting the retroreflectors would reflect back in the direction that they came from (e.g., the UV emitter). UV absorptive material could be placed at, and around, the UV emitters, so that the UV light (after being reflected back to the UV emitters would thereafter be absorbed and thereby reducing UV scatter).

FIGS. 10A and 10B are drawings of a UV absorptive installation according to an embodiment. Shown in FIGS. 10A and 10B, a venue/space 1000 is shown in perspective view and top view, respectively. Venue 1000 could be any possible space that has persons entering and moving through the venue in space and time—and would be a candidate for near-continuous, real-time disinfection process.

Throughout venue 1000 (as shown as an x,y,z configuration), there are UV absorptive strips 1002 on the walls of the venue. As may be seen, such strips may be placed at various heights from the floor and/or ceiling. The placement of absorptive strips at different heights allow for the UV emitters to target various airspaces of the venue at different times and locations. For one example, if a person is standing, so as to obstruct a clear line of fire from one UV emitter at 4 feet from the ground, that UV emitter may change to a strip that is just above the head of the standing person (to say, 6 feet above the ground). When that person sits down, the UV emitter may (e.g., 10 on its next disinfecting pass) then target the strip that is 4 feet from the ground. As mentioned above, markers may be embedded into the absorptive strips that may be detected by various sensors placed in the venue. If a desired number of markers are seen by the sensors, then there is a clear line-of-sight in order to direct UV light.

As may also be seen in FIGS. 10A and 10B, floor strips 1004 may be placed advantageously throughout the venue, so as to provide a disinfecting illumination pattern that can disinfect a desired amount (e.g., 60-90% or any other suitable amount) of the airspace of the venue of a given time period. As may be seen in FIG. 10B, two UV emitters 1006 and 1008 may, at one time, lay down a pattern of disinfecting illumination on a given strip 1004.

The purpose of the UV absorptive strips is multiple. For one example, UV absorptive strips provide a target of the illumination emitters to fire their UV illumination. In such a case. UV scatter is reduced—so that persons in the venue are not subject to more than a desired level of background, ambient UV illumination. Second, if the UV absorptive strips are embedded with a marker (e.g., light, symbol, reflector and/or phosphor that is radiates visible light when impacted with UV light), then the marker may be detected by image cameras throughout the venue.

In the case of a UV-activated phosphor (i.e., phosphors that emit visible light (or other wavelength) when irradiated with UV light), the system may have a level of emitted light from the phosphor that the system expects to detect if conditions are normal. The signal of emitted light from the phosphors may give an indication as to whether: (1) the UV emitter is hitting the strip (or missing it, giving an error indication); (2) something is obstructing the line of fire towards the strip (thus, giving an error indication that some person may be inadvertently in the line of fire, and the emitters may be turned off) and/or (3) the UV emitter is varying in its power level (as thus, giving another possible error indication). It will be appreciated that the present application encompasses UV absorptive layers to be in any desired shape and/or dimensions. For example, the UV absorptive layers may be spots on the venue—either regular or irregular shapes. It suffices that there may be sufficient numbers of spots and/or targets, such that the number of UV emitters that are aiming at the strips/spots can disinfect a desired amount of the airspace in the venue in some pattern of disinfecting illumination.

In one embodiment, entire walls are coated with UV absorptive material. Instead of controlled to fire toward a particular strip, the controller verifies that nothing has been put on the wall in the area to be irradiated (e.g. the occasional picture frame). Such verification may be performed when firing at strips as well. In another embodiment, UV emitters are embedded in flooring and/or lower sections of walls and fire upward or generally upward—toward a ceiling, for example. A power calculation may be performed and if power of illuminations from the UV emitters is sufficiently strong to reflect off the ceiling and reach people below, the ceiling may be coated in absorptive material. Alternatively, the ceiling may be coated with absorptive material even if minimal or no illumination is capable of making it to people below. UV absorptive material may be coated on shipboard hardware, pipes, staircases and other equipment. Nonetheless, in one embodiment, the absorptive strips may be replaced and/or augmented with reflectors. One or more reflectors may replace/augment each strip. The reflectors may comprise a linear array of reflectors. Firing from a UV emitter toward a reflector may be timed such that UV reflected from the reflectors does not impact people or other forbidden objects, much like emitters elsewhere described herein that operate to illuminate spaces and objects around people but do not irradiate people (notwithstanding some exceptions described herein). Each reflector may be treated as a source for calculating reflection trajectories.

In one embodiment, the reflectors are separately moveable (or otherwise aimable) such that a controller may actively position a reflector such that its reflection from one or more emitters does not impact people (e.g., aimed or otherwise falls between people). As such, two emitters or more may simultaneously fire at a single reflector from different angles (potentially widely different angles) if that reflector can be positioned such that both (or all) emissions reflect in safe directions (e.g., away from people—at least not to impact skin, eyes, or loose weave clothing). In one embodiment, the reflectors may be positioned such that reflections are directed into absorptive areas of a room such as painted surfaces, carpeting, ceiling tiles, etc. In one embodiment, the reflectors redirect incoming UV radiation toward one or more ceiling tiles selected for UV absorptiveness. In another embodiment, the ceiling tiles are high enough and specular reflect sufficiently such that any further reflection therefrom does not impact people or are below minimum thresholds established for safety. In still another embodiment, the reflectors are retro reflectors that return UV illumination back toward its source. And in still another embodiment the reflectors direct incident light toward ceiling material having retro-reflective properties and the UV light then reflects back and forth on the clear path until extinct.

In yet another embodiment, the reflectors are positioned (or otherwise aimed) to reflect UV emissions toward a specific target that has been identified as likely carrying a pathogen load (e.g., a touched surface, or a predicted or detected sneeze/cough cloud).

In these ways, rather than being absorbed, UV energy is recycled and employed to kill more viruses and other pathogens. The advantages of the reflective embodiments are at least three fold: (1) increasing irradiation of infected areas/surfaces; (2) lowering system power requirements; and (3) alleviating or reducing the shadow problem by providing multiple additional angles at which objects, surfaces, and spaces may be irradiated.

### UV Walls/Partitions/Fixtures Embodiments

In keeping with the theme of disinfecting populated indoor spaces, there is a need to place a disinfecting partition between people in a work place/warehouse/business and the like. In the context of the present application, the term “UV wall” (or some other disinfecting light other than UV, e.g., microwave, x-ray, infrared) means an illumination field that may assume the shape of a wall or some other containment shape when the illumination is ON. Such UV walls or containment spaces may be affected by reflected light (e.g., MEMS, DMDs, DLPs or other such reflectors or other aiming systems like gimbaling or the like) or by the desired placement of light sources—e.g., a set and/or array of LED lights situated in a strip such that their substantially overlapping light fields from adjacent LEDs assume the containment shape. The wall itself may assume any desired orientation in space—e.g., horizontal, vertical or any other orientation desired. In addition, the wall may substantially be a straight line or any desired shaped curve. The wall may be joined by other UV walls and/or physical barriers to form a bubble or some other containment space.

In many embodiments, the terminus of the disinfecting light in a wall may be a block and/or absorber of the light. In some embodiments, the block/absorber may comprise materials and/or chemicals which advantageously absorb the light; thus reducing the amount of scattering that might tend to raise the ambient level of disinfecting light. The block/absorber may also comprise retro-reflective elements (e.g., corner reflectors, cat-eye reflectors or the like) in order to reflect the light back to the source. In this fashion, the disinfecting light may be used multiple times. In such cases, there may be another block/absorber (with or without retro-reflective elements) near the source to reduce the scatter and/or re-use the light. In some embodiments, the block/absorber may also comprise a set of phosphors that, when absorbing the disinfecting light, emit light in a different wavelength (e.g., visible light). An image sensor placed in the environment may sense the level of visible or other light emitted. The emitted light thus gives a signal to the system that, if the emitted light is of a desired luminance, then the system knows that most if not all of the disinfecting light is hitting the block/absorber. If the luminance is less than expected, then that signal may indicate a fault. For example, a lower emitted luminance may indicate that: (1) something (e.g., a person) is blocking the emitted light (in which case a person may be subject to disinfecting illumination); (2) the disinfecting light is missing the block/absorber and the aiming/steering/situating of the disinfecting lights needs alteration/correction; or (3) the disinfecting light source is diminishing in intensity and the light source may need to be fixed/adjusted. In such a case, a controller receiving this sensor data may instruct the UV lights to turn off. This would also be the case if the sensor are detecting that the UV light is going to be obstructed.

In some embodiments, the wall may comprise a desired thickness of illumination such that pathogen particles diffusing or drifting through it (perhaps by prevailing air currents) affords some statistical range of germicidal effect—e.g., a 95-99% germicidal effect or some other desired range. The desired thickness may also be affected by the intensity of the disinfecting light—that is, if the intensity of the light is higher, then it may be possible to have a less thick wall of illumination in order to get to the desired range of germicidal effectiveness.

In some embodiments, the reflectors may include one or more coatings targeting any of a specific UV wavelength (or wavelengths), ranges of UV wavelengths, sub-regions of any wavelength range, a plurality of discrete wavelengths or sets of wavelength ranges separated by other wavelengths (e.g., separated by a gap or blocking band).

In another embodiment, optional optical coatings may be specifically arranged to enhance transmission of non-visible UV, at specific wavelengths in the short and/or medium UV-C wavelengths (and/or others). In one embodiment, such coatings for optics and/or mirrors are optimized for 2 different wavelengths (e.g., having peak transmissivity and/or low reflectivity for pass through optics, and peak reflectivity without transmissivity for mirrors and other reflectors. In one embodiment, a coating comprises UV transmissivity in one direction and reflectivity in an opposite direction (e.g., a UV one-way mirror) which may be, for example, placed as an entry point for UV into a waveguide.

In yet other embodiments, specific spread functions of neighboring light sources in pathogenic destruction systems affect a desired mixing and/or coverage of UV projected by various individual systems and/or multiple systems operating together. In one embodiment, UV projections are shaped and mixed (e.g., in overlapping illumination fields) in a controlled manner for maximum pathogenic effect. The various embodiments may be advantageously arranged specifically or adapted for use in intelligently aimed/situated pathogenic destruction systems.

FIGS. 11A and 11B are exemplary embodiments/applications of such UV walls. FIG. 11A is drawing of an open office work space 1100 according to one embodiment. The open office is at least partially enclosed by light 1110 at a wavelength (e.g., UV-C, short-wave UV, UV-B, UV-A, Infrared, microwave, x-ray, etc., or mixtures thereof) that is harmful to viruses and/or other pathogens. In many embodiments, it may be desirable to mix in visible light into the germicidal illumination for a variety of purposes—e.g., visible light mixed with invisible light may invoke a blinking reflex to the eye to avoid injury from the invisible light—or otherwise give warning that an area or person may be illuminated. Light may be emanated from structures, such as strip 1120 containing, for example, UV sources 1125, and is referred to hereinafter as a UV wall or UV wall, enclosure, or other structure regardless of the wavelengths or combinations of wavelengths contained therein. A strip or structure corresponding to a set of sources is not required but convenient to maintain spacing of the sources, provide a structure to mount controllers (generally shown at 1102), sensors (generally shown as 1104), etc., as desired for a particular installation. The strip structure is also convenient for retrofitting offices or other buildings with a UV enclosure or a UV wall at desired locations. Such desired locations include around office structures, frequently visited areas, doorways, eating areas, restrooms, etc.

Sensors, which may be installed on the strip or elsewhere on other structure throughout the space/venue or may be remote connected sensors, or wearable sensors, mobile devices, etc., may provide location data of people and other objects to be avoided with illumination (“forbidden” object) that is transmitted to a control (1102) that turns off the UV wall when any such objects approach or enter the area in which the UV wall is operating. The closeness of a forbidden object that triggers turning the UV wall off can be variable depending on a number of factors including, but not limited to, a risk level associated with the object, a speed of the object, a potential speed of the object, the season, current pandemic or local outbreak status(es), and/or other data. It should be appreciated that controller 1102 and sensors 1104 may be located at or near the light sources, reflectors (if any), gimbals (if any)—or may be distributed throughout the venue/space/environment. In the case of the controller, the controller may be remote from the venue/space/envimnment and may be hosted in the cloud/internet (e.g., as a SaaS).

In one embodiment, a series of UV walls may be placed perpendicular to an assembly line (or other workspace), each UV wall separating one worker from the next in a side-by-side (or shoulder-to-shoulder arrangement). Such an arrangement may be utilized in, for example, meat processing plants, fulfillment centers, and other locations where workers are subject to high rates of infection from nearby co-workers and potentially the products themselves.

As illustrated in FIG. 11, an opposing structure(s) (or terminus) 1130 and UV sources 1135 are shown at or near a ceiling area of the office and define a height of the UV wall. The UV sources 1135 are not required. To increase efficiency, the opposing structures 1130 may include a UV reflector configured to reflect UV light back into the UV wall, in effect, re-cycling the projected UV lights. In one embodiment, no opposing structure is required. In another embodiment, the reflector(s) is replaced with UV absorptive material(s). In yet another embodiment, reflective structures (e.g., corner boxes) are constructed over a UV absorptive layer. In yet another embodiment, the reflective structures vary in tilt from essentially perpendicular or normal configuration to an angular tilt (off normal, toward UV source(s)) at outer portions of the reflector, and such variation may be sectioned and/or transition smoothly toward the outer portions (e.g. edges).

Strip 1120 may also include reflector(s) thereby enabling further re-cycling of the returned wavelengths (and reflection of UV from UV sources 1135 if present). Sources 1125 and 1135 are shown aligned, but may be offset from each other and may be spaced differently than as illustrated and may include variable or custom distances between the sources and may utilize other arrangements as described elsewhere herein. Such desired distancing may be chosen to affect an overlapping field of illumination and/or a unbroken chain of illumination at both ends of the UV wall, for example.

The UV sources may comprise laser sources, LEDs, and/or more traditional bulbs (coherent and/or incoherent sources), for example. Each of the sources may be paired with lenses, films, or other optics to modify a character or shape of the projected light. The reflectors may likewise take many forms including retro-reflective mirrors, plain UV mirrors, partially dished or parabolic reflectors, each including various types of structures and layers and/or coatings. Similarly, various optical elements and covers (e.g., a cover over strips 1120 to protect from foot traffic) may include various types of structures, layers, and/or coatings.

FIG. 11B is a drawing of a UV extended partition 1180 according to an embodiment. The UV extended partition 1180 is mounted, for example, between two seats. The partition may be constructed of Plexiglas or other clear material or it may be opaque. The partition provides a barrier between two seats such as on an airliner, bus, or in a theater, for example. In the illustrated embodiment, a top edge 1181 includes UV light sources 1182, 1183, and 1184. In this example, each of the illustrated light sources has a different projection, namely a different spread function to produce a UV wall as an extension to the top edge 1181. The UV wall/extension may be directed to a reflective device (or otherwise a terminus) 1186 which may be, for example, a structure with a UV reflective coating such as one or more of the UV reflective coating described elsewhere herein. Additional sources (e.g., sources 1185) may be provided on the reflective structure or nearby and may be configured to provide filler irradiation in UV gaps between the sources 1182-1184 (projected to the top edge 1181 between sources). Other additional sources or optics may be placed on or along top edge 1181 and utilized to fill the gaps from the top edge itself. As will be appreciated by the skilled artisan, many different arrangements of sources or optics may be utilized and any alternative selection configured to produce a same or similar result is believed apparent to the skilled artisan in light of the present disclosure.

FIGS. 12A and 12B depict two embodiments of UV walls made in accordance with the principles of the present application.

FIG. 12A is a drawing of a dished reflector 1200 according to an embodiment. The reflector comprises a first reflector 1210 and an opposing or second reflector 1250. The first reflector 1210 illustrates exemplary UV sources 1215 placed in the reflector/strip that may be LEDs, diffused lasers, laser arrays, electrical bulbs or other sources emitting UV. The sources may include diffusers, polarizers, enhancement films etc. that may be configured for various purposes including, for example, producing a desired spread function or mixing with neighboring sources. The emitted spread functions may vary by location on the reflector, such as, for example, sources at an end of the reflector (compared to those in the middle) may include a spread function that may, for example, favor projections in a direction that provides a substantially more consistent (or, at points, more concentrated) illumination of the opposing reflector 1250 and/or the airspace between the reflectors.

In the illustrated embodiment, the first reflector 1210 is a source device having UV sources 1215. In this configuration, the sources project UV toward the opposing (or second) reflector 1250. An advantage realized as such is that simulations of clouds of potentially pathogenic material (e.g., discharge aerosols) generally move from a human source (cough or sneeze) with air currents which are usually up and over barriers such as partitions, seatbacks, etc. In the embodiment where the sources in a bottom-up configuration, the UV light might be strongest in areas closest to the source and pathogens moving up and over a bottom-up configuration are closer to the sources and therefore irradiated more intensely (comparted to other portions of the projection(s)) and more likely to receive a “kill” dose of UV.

While illustrated as a bottom-up configuration, both reflectors may comprise sources. In one embodiment, the top or opposing reflector 1250 may include UV sources. In one embodiment, the opposing reflector may include sources having a more narrow spread function than the bottom sources. The more narrow spread function may be, for example, configured to illuminate spaces between the sources on the other reflector. With a more narrow spread function, the UV traveling to the other structure may maintain more of its energy and increasing its chances of delivering enough power to kill or deactivate pathogens. In this manner, a more active area near the structures may include more power. It should be appreciated that the opposing reflector arrangement may be placed in any desired orientation—e.g., side to side (horizontal) or however desired.

In one embodiment, one reflective structure has a source or sources having a first projection described as UV W degrees angular projection and an opposite structure includes a second projection described as UV N degrees of angular projection, where W degrees may be greater than N degrees. One embodiment, illustrated in FIG. 12B, is a drawing of an embodiment including different spread functions on opposing structures. A narrow upper beam 1280 is projected into a gap between two lower sources (of wider beams, e.g., 1285) effectively eliminating the gap. In one embodiment the spread function or angle of projection amongst the several light sources may vary, which may include, for example a projection angle of a wider beam, e.g., 1285, adjacent to another projection, e.g., 1286, that spreads uniformly into the wider beam on the adjacent side, and a slightly more narrow (or wider) spread on the opposite side (such as to match the projection beam to a structure, for example). Yet other embodiments include additional optional sources, for example more of the same sources, smaller filler sources between main sources, or cross illumination sources (e.g., cross sources 1290, 1292) which may be utilized to remove UV gaps (or add strength to the irradiations—particularly with respect to a second structure which may encounter higher pathogen traffic. Nonetheless, it should be understood that any such embodiments may be practiced where the sources and/or structures are constructed equally or in an opposite fashion (e.g., cases where upper structures (or any side or oblique structures in non-vertical walls) are the high traffic area due to air flow or other issues.

The structures include reflective materials, such as reflective plate 1225 and 1255 (not in view). In one embodiment, the reflective plates may comprise an optical structure coated with a UV reflective material. Any of the embodiments or compositions of reflective materials discussed herein or any equivalent materials of similar or different structures having good performance in the UV regions may be substituted therewith. Parabolic reflectors 1230 may surround the plates and any sources and may similarly include UV reflective coatings. Reflective plates 1225 and 1255 may comprise retro-reflectors. In one embodiment, both reflective plates comprise retro-reflectors and may trade re-cycled UV beams back-and-forth until extinct. In other embodiments, reflective plates 1225 and/or 1255 may further comprises phosphors 1205 (e.g., either discrete spots or in a strip or other desired arrangement). Phosphors 1205 may emit visible light when absorbing disinfecting light. If a sensor is detecting the luminance of emitted light, then the system may be able to detect working and/or fault conditions, as discussed above.

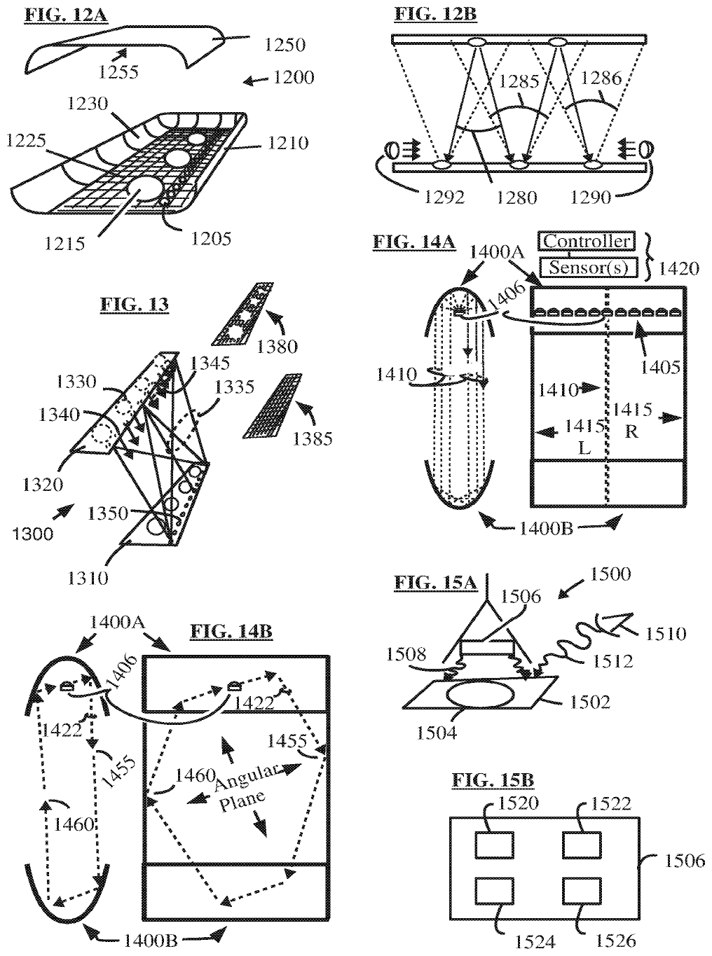

FIG. 13 illustrates another embodiment of a UV wall 1300 having an object detection safety turn-off mechanism. UV sources 1330 which may be, for example, a UV die or substrate comprising one or a plurality of UV LEDs, may be spaced at intervals along structures 1310 and 1320 or provided in a contiguous row. Optional sources 1340 (e.g., sources on an upper structure in this embodiment) project in a desired direction (e.g., downward as shown by light 1345) and/or at angles (e.g., 1335) to corresponding detectors 1350 on an opposing structure (lower structure 1310 in this embodiment). The detectors 1350 may be, for example, connected to a controller such that when not receiving the intended illumination the controller turns off the UV sources (e.g., nearby UV sources or all of the UV sources). Alternatively, different types of sources and or detectors may be utilized to notify the system of an incident or approaching object upon which the UV sources may then be turned off.

In the illustrated embodiment, both first and second structures are shown having UV sources 1330, generally pointed toward each other. However, in alternative embodiments, only one of the structures has UV sources and may include laser or other detection systems for turning off the UV sources such as cameras—or as discussed in more detail at various other places in this disclosure. For example, in one embodiment, the second structure is replaced with a prismatic material, structure, film, or sheet 1385. The prismatic material increases efficiency by reflecting un-absorbed UV back to its source. Alternative structures may include any of prism variations, box corner elements, concave troughs, parabolas, ridges, and the like, and, generally speaking, such structures may be repeated and continuous when embodied in/on a sheet or film. The UV emitting structure may also include a prismatic or other structural mask 1380 to enhance further reflections (e.g., retro-reflectivity) which may then be “re-cycled” (e.g., re-reflected) multiple times until extinct.

FIGS. 14A and 14B are yet other embodiments of UV walls. FIG. 14A comprises parabolic troughs 1400A (e.g., upper) and 1400B (e.g., lower) are juxtaposed and LEDs 1405 are placed in the upper or lower troughs (shown in upper, but optionally placed in the bottom, mixed upper and lower, and/or side or angularly mounted configurations—not shown). One plane of illumination 1410 is illustrated, but it should be understood that a plurality of planes at all angles emit in a 360 degrees around the illustrated LEDs.

The LEDs are positioned, for example, to emit toward a vertex of the parabolic trough. Other illumination arrangements may also be utilized. Optionally, a light pattern of one or more of the LEDs (or other light sources) may be provided by design to emit in one or more portions or specifically engineered planes. The light sources may be non-LED sources such as tubes, lamps, etc. The light sources (e.g., LEDs) may be, for example, one or more of UV-C, UV-B, UV-A, and blue light LEDs. A combination or pattern of LEDs may be utilized to provide desired amounts of each of desired wavelengths in the wall.

Optional edge reflectors 1415R and 1415L are positioned along sides between the parabolic troughs and reflect illuminations that would otherwise exit the wall area (between the two troughs) back into the wall. Controllers and sensors 1420 detect incursions and/or approaching and/or potential incursions into the wall area and the wall may be turned off or segments/portions of the wall may be turned off in response.

FIG. 14B is a diagram of an angular plane of illumination in the UV wall that is at an angle to the single plane 1410. The angular plane of illumination (across 1422, 1455, 1460) extends down the trough and is reflected off edge reflector 1415R (e.g., at 1455) into the lower trough (1400B), for example, and ultimately returning to the upper trough 1400A (e.g., via reflection and may be recycled multiple times between the parabolic troughs and edge reflectors.

The multi-plane nature of the light emitted from the LEDs and maintaining their emissions in the wall and recycling via the parabolic troughs and edge reflectors makes a relatively high intensity and efficient wall of UV light. The wall may be utilized, for example, to divide an establishment into sections, help prevent cross-contamination of viruses and/or other pathogens, provide an extension of a physical barrier (such as a cubicle wall, check-out screen, bank window, divider between seats in a theater or on an airplane or other vehicle, etc.), and/or maintain a UV wall around a work area to kill or deactivate viruses and other pathogens coming into or leaving that work area.

In other embodiments, specific light fixtures are herein described. In particular, it may be desirable to have cleaning protocols and/or lighting modules that affect a multi-mode functionality. In one embodiment that cleans surfaces, the system could apply more than one wavelengths of light to affect a more thorough cleaning. FIG. 15A depicts one such embodiment of multi-mode cleaning. Space/venue 1500 may comprise surfaces 1502 that occasionally need cleaning (e.g., after someone has touched the surfaces at spot 1504). Lighting fixture 1506 could be positioned above the surface (e.g., as a lamp fixtures or the like). Multiple and different wavelengths 1508 may be employed to clean the spot 1504. For example, UV light may be mixed together with infrared, microwave and/or x-ray to clean the surface in one light source 1506. Alternatively, light source 1506 may emit one set of wavelengths (e.g., UV), while a second light source 1510 may emit a second set of wavelengths (e.g., infrared, microwave and/or x-ray) in a beam 1512 to clean the spot.

In another embodiment, a liquid may be applied to spot 1504 that may interact with the various light wavelengths in order to affect a deep clean. In one embodiment, water may be applied to spot 1504 and then infrared and/or microwave light may be applied to create steam, which may have a cleaning effect. In or around the same time, UV light may also be applied to provide additional germicidal effect—particularly, if the steam may create an aerosol containing pathogen particles. For another example, alcohol (either ethanol and/or methanol) may comprise the liquid—which has its own germicidal effect and vaporize with less light applied than water.

In other embodiments, the system may interact with staff persons in the venue/space. For example, the staff may inquire of the system (e.g., through some interface, like typing or voice activated) what surfaces need to be cleaned. The system may—e.g., through monitoring the space over time with image cameras what surfaces have been touched—show the staff what surfaces to be cleaned by shining visible light (e.g., red laser light) the spots on surfaces to be cleaned. The staff may then spray the liquid on the spots and then inform the system to start cleaning. The system may then apply the multiple wavelengths of light, as described above, to start the cleaning process.

In other embodiments, a single light source may comprise the multiple wavelengths in one lighting package. FIG. 15B depicts lighting fixture 1506 as comprising multiple sources (e.g., different LEDs). For example, fixture 1506 may comprise a white light source 1520, an infrared and/or microwave source 1522, a colored lights source 1526 and/or a UV source 1524. In one embodiment, light fixture 1506 may be a lamp or lighting fixture over a surface (e.g., a table, countertop or the like). In one embodiment, if people are located at or near the surface (e.g., sitting at a table), then fixture 1506 (under controller command wherein the controller is receiving sensor data in the venue, detecting the presence or absence of people at the table) may emit white and/or colored light to provide a pleasing lighting environment for the people. After the people leave and the surface needs cleaning, then the white light (which may comprise a package of red, green and blue LEDs) may switch to a colored light, like yellow, to indicate that the surface is about to be cleaned. That may involve staff to spray the surface with such liquid as mentioned above. Fixture 1506 may then apply optional infrared and/or microwave light to interact with the liquid—while possibly at the same time switching the visible light to red to indicate germicidal illumination happening. At or near the same time, fixture 1506 may apply UV light for a deep cleaning. After the germicidal cleaning is complete, the fixture may switch to green or white light to indicate that the cleaning is finished.

### UV Walls for Medical Applications

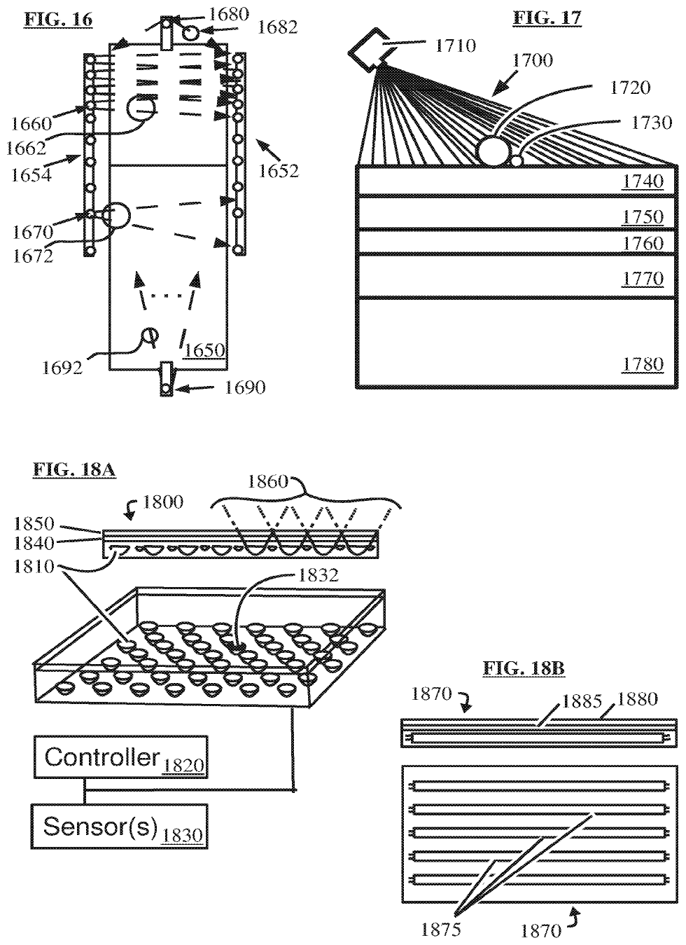

It has been known for nearly a century that UV light disinfects surfaces in operating rooms. Typically, there might be an overhead UV sources that directs UV light downward from the ceiling to the operating theatre (while it is devoid of people); thus sterilizing the theatre. Described herein, a novel concept of UV walls can literally be turned on its side (e.g., that is to say, substantially horizontal and/or parallel to the plane of the patient) to create protection for medical staff and personnel (e.g., EMT, nurses, doctors, etc.) that are caring for patients who may be contagious with a respiratory infection—or alternatively, to prevent a patient having a medical procedure from acquiring a Hospital Acquired Infection (HAI). FIG. 16 is one embodiment of an operating table, hospital bed, gurney, wheelchair, or other suitable medical structure like, which comprises a set of UV sources that are placed around the edge of the structure that provides a UV wall of protection over the relevant part of the patient—and possibly, intervening between the patient and the healthcare provider.

For merely one example, FIG. 16 is a drawing of a gurney or hospital bed 1600. The gurney may be configured to comprise UV emitters at various locations. The UV emitters are configured to cast UV light encapsulating a patient or portions of a patient. For example, a head area of a patient may be encapsulated in a globe or box of UV irradiation (“UV bubble”). Perhaps most importantly, the UV irradiation provides a protective layer that prevents healthcare workers from coming in contact with raw unattenuated viral or pathogenic content expelled directly from the patient, e.g., via respiration.

In one embodiment, gurney 1600 may comprise a UV emitter/source/module 1680 at the head portion of the gurney. UV source 1680 may be placed on top of a support structure/bar 1681 to provide clearance of the head of the patient (not shown) when lying down. UV source 1680 may emit an arc of illumination 1682 around the head of the patient. This arc may be produced in any number of ways—e.g., via a bank of UV emitters (such as LEDs) or may be reflected by any reflector embodiments mentioned herein. Arc 1682 may have a desired level of thickness such that, in combination of the intensity of the UV emission, gives a sufficient amount of statistical pathogen “kills”. For example, if lower powered UV sources are employed, then the thickness of arc 1682 may be lesser than if more powerful UV sources are used. It may suffice that the thickness of are 1682, together with the power of UV sources give a 90-99% probability that expired viral/pathogens from the patient are “killed” prior to leaving the UV bubble. Other effective kill ratios (lower that 90%) may be suitable—e.g., depending on how much pathogen load is modeled to create serious illness or health consequences.

In yet another embodiment, the terminus of the arc of illumination may be in a barrier that is placed at the head, neck, torso or other part of the patient. Such barrier may be a substrate that has a portion and/or coating of a UV absorptive material. It may also comprise a set of retro-reflectors that may reflect UV light back to the UV source and re-use the UV light. There may also be a UV block at the UV source in such embodiments to prevent unwanted UV illumination or scattering. In another embodiment, the patient may have draped over him/her a UV “bid” or otherwise cloth may comprise UV block/absorptive material, with or without a set of retro-reflectors. The patient may also be given a set of protective eyewear/glasses/googles that protects the patient's eyes from UV illumination.

In another embodiment, safety bars 1610 and 1620 are locations where UV emitters may be situated. The safety bars may be moved forward on the gurney to provide better positioning, and the safety bar may be raised in height to help clear a patient's body and/or facial features. Alternative locations on the gurney or structures attached to the gurney, such as posts, poles, bars, etc. (not shown) may provide other locations from which to project UV.

FIG. 16 is a drawing of an inside view of an exemplary implementation of an embodiment of a gurney safety bar. The safety bar illustrated comprises emitter locations 1612 each of which provide a UV emitter which may be embodied as any one or more of a UV laser, UV diode, UV laser diode, xenon lamp, pulsed xenon lamp, etc. The emitters may comprise aiming mechanisms such as a mirror, modulators, or louvers (e.g., LCD louvers), for example.

The UV emitters may be positioned next to each other or separated by reflective structures or material that may increase the amount of UV coverage by reflection. Reflection may be back toward the source or skewed to spread coverage over a broader area. Gurney safety bar may have, for example, a corresponding offset structure. In one embodiment, the inside of the safety bar is a flat (or concave reflective concentrating structure) e.g., a polished metallic surface and may comprise one or more UV enhancing or reflective coatings. Alternatively, the spaces between the emitters may be coated in UV absorptive material reducing reflections that might otherwise increase ambient UV levels.

FIG. 16 also shows exemplary UV module or emitter locations and illumination (irradiation) patterns. As shown, safety bars 1652 and 1654 have emitters positioned so as to be directed at each other, which may take advantage of direct illumination of a corresponding emitter's reflective structure (if any). The emitters may also be configured in an offset pattern such that an emitter's direct path meets a reflective or absorptive portion of its opposing safety bar. In one embodiment, electronics or other safety mechanism may prevent operation unless the safety bars are raised to a same height.

The emitters may have a field or spread pattern such as patterns 1662 (relatively narrow) and 1672 (relatively wide). The emitters may be spaced at different interval densities, such as close together near a head or more pathogenically active areas, and spread apart more (or absent) at other portions of the patient's body. Cross irradiation may be provided at a head of the gumey, e.g., emitter 1680 located on a head post, and emitting a UV beam (e.g., beam/arc 1682, relatively wide) across or in direction(s) mainly perpendicular to UV emissions from the safety rail. Cross irradiation may also be provided at a foot post (e.g., emitter 1690), and corresponding beam 1692 (relatively narrow beam, shown as an example). Downward UV beams may also be provided, for example, from the safety rails or any of the posts or other structures thereby completely encapsulating the patient.

Cameras or other detecting mechanisms, as described herein, may also be utilized in a fashion similar to that discussed above for the UV face shield. Other detecting mechanisms applicable may be laser sensing projected over and under the UV light paths which, when broken turn the UV projections off. Intelligence built into a system energizing the UV modules may interrupt emissions when any object not to be irradiated enters any of the UV paths, again, similar to that discussed above. In one embodiment, of particular usefulness for performing pathogenically risky procedures, such as an intubation, the system may be set to an always-on mode regardless of UV path interruptions. Such a mode is, for example, intended for uses of short duration where participants are suited in UV and pathogenic PPE (Personal Protective Equipment) which may also utilize a UV face shield as described above.

### UV Disinfecting Table Preparation Embodiments

In the area of food preparation, the following embodiments are disclosed that utilize UV light—either passively, actively or in combination—on preparation surfaces to kill pathogens (e.g., bacterial, viral or the like) while making food/meals and the like. In one embodiment, a table top (which may be a work table, conveyor belt segment, or any surface subject to contamination) is constructed with a UV transparent layer over a reflector, reflective materials or structure that allow UV energy applied from a source to pass through to the reflective layer and reflect in directions that irradiate microscopic (or larger or smaller) shadow areas on the table surface. The table may include a diffusion layer such that UV energy is scattered in all directions such that when reflected back toward the table surface the shadow areas are illuminated. Such diffusion tends to overcome the problem of when UV light fixtures send UV toward surfaces in a room, disinfection can be impeded by UV absorbing objects that are located on or near the surface and cast “UV shadows”. In other words, very small UV-opaque “shadow casters” might remain and block UV from reaching bacteria or viruses.

In other examples, microbes may be able to hide out in crevasses or other irregularities in the surface that provide adequate shadowing that allow the microbes to escape irradiation. In either cases, the new composite structure has been devised in order to solve that problem. FIG. 17 is one embodiment of a UV disinfecting food preparation table 1700. For merely illustration purposes, ball 1720 is representative of the irregular surface (e.g., a crevice, or other imperfection) of any table that provides shadowing protection to a microscopic pathogen (illustrated at ball 1730). As can be seen, pathogen 1730 is hiding in the shadow of imperfection 1720 and will not be “killed” by UV light emitted from source 1710. The idea is that light from UV source 1710 will penetrate a few one or few surfaces of the table and be reflected by a diffuse UV reflective layer—so that some UV light rays will reach pathogen 1730 from below.

Layer 1 (1740) comprises a top layer (e.g., perhaps small layer approximately 0.1 mm thick or any other suitable large or small layer) of a material such as Corning Willow Glass which is flexible, unbreakable, durable and UV transmitting, Corning Willow Glass is a lamination to polymeric substrates to make them scratch resistant and easily cleanable. In addition, Layer 1 may comprise quartz or mica (e.g., thin enough to be transmissive/translucent) or any other suitable UV transmissive layer.

Optional Layer 2 (1750) comprises a reasonably inexpensive UV transmitting material, which may be a form of RTV silicone (as noted in optical characterization of RTV615 Silicone Rubber Compound” by W. Li and G. M. Huber, doi:10.1088/1748-0221/9/07/P07012 and herein incorporated by reference). In one embodiment, it should be thick enough (e.g., several millimeters) so that the UV light it transmits will shift considerably in the lateral direction during its largely unimpeded travel within the material.

Layer 3 (1760) comprises a diffuse UV reflector. It may comprise a bumpy surface coated with a thin metallic film such as aluminum, rhodium, platinum or silver that may itself be scratched or otherwise roughed. In yet another embodiment, layer 3 may be to deposit a layer of material that reflects UV light (e.g., white sand, white pigment, or mica (e.g., thick enough to be reflective) or any other suitable UV reflective material—such as detailed in the literature, e.g., “Ultraviolet Reflector Materials for Solar Detoxification of Hazardous Waste” by Gary Jorgensen, Rangaprasad Govindarajan, https://www.nrel.gov/docs/legosti/old/4418.pdf, and herein incorporated by reference). In other embodiments, Layer 3 may comprise a UV reflector material made from an aluminized polymer film (e.g., SA85 from 3M Company) having a front surface protective coating without appreciable UV absorption. In yet another embodiment, Layer 3 may comprise a multilayer dielectric interference coatings. Typically, quarter-wave-thick coatings of high index of refraction materials (such as Al2O3, HfO2, ZrO2, and Sc2O3) are alternated with low index layers (for example, SiO2, CaF2, and MgF2, which are also quarter-wave thick.

Ten to fifty layer coatings may not be unusual.

In another embodiment, the bottom portion of Layer 2 may comprise a layer of highly doped non-UV absorbing white pigment (e.g., any common white pigments, white sand, etc). Layer 2 might also comprise white sand particles that are known to reflect UV light.

Finally, optional layer 4 (1770) comprises a strong table substrate that makes the sheet as a whole strong and easy to handle, machine, and install. Examples could include polycarbonate, acrylic, etc. Layer 1780 may be any suitable substrate to support the above-mentioned layers.

In another embodiment, the boundary between layers 2 and 3 may not be sharp, and in fact it may be practical to combine them into a single layer with an intermediate level of white pigment doping. Several features include: (1) most of the incoming UV rays end up returning back through the top layer of Willow glass, (2) they are reflected largely diffusely, and (3) during the reflection process, they shift laterally by a great enough distance that they irradiate, from multiple angles, any shadowed pathogens. Another feature is that the typical distance of lateral displacement exceeds the typical size of the cast shadows. A further advantage of this approach is that it may eliminate the need to irradiate a given surface from multiple directions (which is yet another embodiment to minimize the shadow casting problem), thus reduce the possible number of UV sources.

For many of the uses envisioned herein for the reflective layer, there is no particular need to reflect UV light in a specular fashion and in that case less expensive diffuse reflector systems may be employed. An efficient diffuse reflector can be made in the fashion employed in white paints and also white materials such as paper. In one embodiment, it may be desirable to incorporate a number of dielectric interfaces within materials that are quite non-absorptive. In such systems, each individual interface scatters a small portion of the incident light, and collectively, they cause most of the light to reflect back, diffusely. In addition, it may be desirable for the materials in the system to be fairly non-absorptive for the specific band of radiation involved. For example, a common system for paint involves white particles in acrylic polymer.

However, there are many alternatives. In the UV band, for example, a UV transparent RTV silicone material could be used as the matrix, and similarly other pigment materials could be useful, possibly including microscopic air inclusions. It may be desirable to have a sufficient refractive index difference between the two materials, and a desired number of optical interfaces, whereby the structures may be small, numerous, and substantially non-absorptive at the wavelengths of interest.

While FIG. 17 depicts one embodiment of a UV disinfecting table top surface that employs passive diffuse reflection of external UV light sources, other embodiments herein describe both active UV disinfecting tables and a hybrid of both passive and active UV illumination.

FIGS. 18A and 18B are two embodiments of a UV disinfecting table surface for food preparation—or any other reason for disinfection is needed to prepare items.

In FIG. 18A, an exploded view of a table top (which may be a work table, conveyor belt segment, or any surface subject to contamination) is shown that may comprise active lighting elements or other light sources underneath a UV transparent coating or surface. The lighting elements may be an array of individually modulatable UV LEDs. In one embodiment, the LEDs or other lighting element could be placed normal to the surface, illuminating the surface directly.

UV LEDs 1810 are placed into a table substrate and may be encapsulated by a UV-transmissive material 1840 (quartz, Willow glass, silicone, mica etc.). The LEDs may be either UV-A, UV-B UV-C or VUV (vacuum), or others. In one embodiment, the array of LEDs of various wavelengths (e.g. UV-A, UV-B, UV-C, VUV) may form some repeatable unit comprising the table surface.

The LEDs may be controlled by signals from a controller 1820 that modulates the intensity of light according to controller processing. The controller may take as input sensor data. Sensors 1830 may be placed adjacent to the LEDs—for one example, see sensor 1832—other sensors may be placed across the surface to provide image data of the table surface immediately above it. In operation, sensors may monitor certain conditions, such as the ambient light immediately above the sensor, the color of meat as it is exposed to UV light, etc. In one embodiment, the controllers may energize the neighboring LEDs where sensor detect a suitable decrease in ambient lighting (e.g., indicating that meat and/or other food items are above the LEDs; hence the need to irradiate/disinfect the food). Once the food items are removed from the table, the sensors would then detect and increase in ambient lighting and the controller would command the neighboring LEDs to OFF state, thereby keeping the workers from unnecessary UV exposure.

In addition, the sensor may detect any undesired changes in the color of the meat (e.g., indicating denaturing of meat protein). This condition may result in the controller modulating down the intensity of the UV illumination or turning it OFF. A top surface 1850 may be made of UV transmissive, durable material—such as Corning Willow Glass, quartz, mica or the like. The top surface should be durable enough to withstand knives and other utensils being used on the surface.

In one embodiment, VUV LEDS may be hermetically sealed—their light would kill pathogens in direct contact with surface. One advantage of VUV is that it kills effectively and once the light leaves the surface, its transmission is blocked (or significantly reduced) by air. This may reduce the need for protective gear for workers.

VUV may also create ozone, which, at the surface of the table may be desirable for its additional pathogen killing capability. If desired, optional fans and ducting may be configured to remove excess ozone.

In another embodiment, UV-C may shine down (but not hitting people—directed by intelligence away from people or aimed at non-populated areas or equipment). UV-C may be used to convert any ozone created by VUV LEDs back to O₂.

In yet another embodiment, a combination of the above reflective table or coating using a reflective/scattering/diffusion layer and the embedded UV LEDs may comprise the work station. In this embodiment, a separate UV light source could illuminate the table surface, having its light reflected to hit microbes in the shadows and the embedded UV LEDs may directly irradiate the microbes themselves.

In yet another embodiment, a combination of VUV (which kills pathogens), its produced ozone (which also kills pathogens), and UV-C (which kills pathogens) and creates O₂ back from ozone and the reflective layer techniques to foiling shadow.

FIG. 18B is another embodiment of an active UV disinfecting table 1870, as shown with a side view and a top view. In top view, it may be seen that the table substrate has a number of UV lamps 1875 emplaced. A UV transmissive LCD layer 1885 may be placed on top of the UV lamps and a final UV transmissive durable layer 1880 tops the structure. Sensors embedded in the table structure (e.g., around the UV lamps or elsewhere).

In operation, if the sensors detect a drop in ambient light, then it is assumed that there is something in need of disinfection and the LCD cells near the sensor would open and allow UV light to transmit through the table structure. Otherwise, the LCD cells would remain closed and no UV light is allowed to transmit to the surface of the table.

### Enumerated Embodiments

The following enumerated embodiments are presented to illustrate certain aspects of the disclosure, and are not intended to limit its scope. The use of the word “includes” and/or “including” encompasses the meaning of all of the following: “comprising”, “consisting of”, “comprising essentially of”, and/or the like.

A first enumerated embodiment includes: a set of UV emitters placed in an indoor venue; a set of sensors detecting objects in the venue according to at least one characteristic; a controller, the controller receiving sensor data and outputting control signals to the set of UV emitters; the control signals modulating the set of UV emitters in at least one characteristic such that UV light does not substantially illuminate living beings in the indoor venue.

A second enumerated embodiment includes any of the first enumerated embodiments, further including: at least one reflector UV emitter that includes a UV light source, the UV light source directing its light to a reflector, the reflector including one of DMD, MEMS, LCoS and grating.

A third enumerated embodiment includes any of the first through second enumerated embodiments, further including: a visible light source such that visible light is mixed with UV light to give an indication of where UV light is being applied.

A fourth enumerated embodiment includes any of the first through third enumerated embodiments, further including: control signals to UV emitters to effect one of the following: turn ON/OFF, modulate intensity and PWM the energy output of the UV emitters.

A fifth enumerated embodiment includes any of the first through fourth enumerated embodiments, further including: the set of sensors including at least one of the following: image/video cameras, stereo vision, LiDAR, radar, heat/IR sensors, ultrasonic sensors, UV photodetectors and audio microphones.

A sixth enumerated embodiment includes any of the first through fifth enumerated embodiments, further including: the controller determining from the sensor data whether a UV emitter has a clear line-of-sight to send UV light in a given direction.

A seventh enumerated embodiment includes any of the first through sixth enumerated embodiments, further including: controller determining from audio data whether a person in the venue has sneezed and sending disinfecting light to the sneeze cloud.

An eighth enumerated embodiment includes any of the first through seventh enumerated embodiments, further including: at least on direct UV emitter, the direct UV emitter including: a UV light source, a UV transmissive lens.

A ninth enumerated embodiment includes any of the first through eighth enumerated embodiments, further including: a UV light source including a set of individually controllable UV LEDs.

A tenth enumerated embodiment includes any of the first through nineth enumerated embodiments, further including: a projection lens including UV antireflective coating.

An eleventh enumerated embodiment includes a method for setting up any of the first through tenth enumerated embodiments further including: identifying fixed and movable objects within the venue; providing a spatial model of the venue with the identified objects; and associating particular cleaning protocols for specific objects in the venue.

A twelfth enumerated embodiment includes a method for setting up any of the first through eleventh enumerated embodiments further including: initialing using visible light to test beam steering for safety.

A thirteenth enumerated embodiment includes a method for any of the first through twelfth enumerated embodiments further including: a heat sensor, the heat sensor detecting a person who is running a fever while in the venue and sending an alert to staff.

A fourteenth enumerated embodiment includes a method for any of the first through thirteenth enumerated embodiments further including: employing the most rigorous cleaning protocol of the venue when the space is devoid of living beings.

A fifteenth enumerated embodiment includes a method for any of the first through fourteenth enumerated embodiments further including: projecting a set of visible colored light, each color to indicate a different operating mode of the system.

A sixteenth enumerated embodiment includes a method for any of the first through fifteenth enumerated embodiments further including: identifying the surfaces within the venue that are in need of disinfection; determining whether there is a clear line-of-sight between the UV emitter and the surface; and irradiate the surface if there is a clear line-of-sight.

A seventeenth enumerated embodiment includes a method for any of the first through sixteenth enumerated embodiments further including: detecting data that are associated with the a pathogen cloud event; determining the location of the pathogen cloud event; and applying disinfecting radiation to the area of the pathogen cloud event.

An eighteenth enumerated embodiment including any of the first through seventeenth enumerated embodiments further including: UV sources feeding UV light into an optical multiplexor, the optical multiplexor feeding UV light into a set of optical light guides wherein the optical light guides feed UV light to a set of reflector UV emitters in at least one venue.

A nineteenth enumerated embodiment including any of the first through eighteenth enumerated embodiments further including: hosting the controller remotely from the venue, the controller receiving sensor data from at least one venue and the controller sending control signals to UV emitters in at least one venue.

A twentieth enumerated embodiment includes a method for any of the first through nineteenth enumerated embodiments further including: a controller, the controller sending health pings to sensors throughout the venue; the controller executing AI/ML routines to classify objects as living/non-living; the controller predicting the motion of objects; and the controller scheduling a cleaning of one of a desired airspace and surface in the venue.

A twenty first enumerated embodiment includes a method for any of the first through twentieth enumerated embodiments further including: performing a system check of at least one sensor in the venue; determining whether there is a clear line-of-sight for UV light to potential target; determining whether potential target is not in an exclusion zone; determining there has been a desired amount of time when the last disinfection of the potential target has transpired; and commanding UV light to the potential target, if all conditions are passed.

A twenty second enumerated embodiment includes a method for any of the first through twenty first enumerated embodiments further including: determining Areas of Inclusion (AOI) in a venue; determining Areas of Exclusion (AOE) in a venue; detecting and classifying objects in a venue; and predicting if moving objects will occlude a clear line-of-sight path for UV light to a potential target.

A twenty third enumerated embodiment includes a method for any of the first through twenty second enumerated embodiments further including: for at least one camera sensor in the venue, partitioning the image frames from the camera sensor into blocks; capturing a baseline image frame; computing the difference between the baseline image frame and a subsequent image frame on a block by block basis; determining blocks for any changes in at least one desired metric; for any block having a threshold change in at least one metric, turning OFF any UV light directed to that block.

A twenty fourth enumerated embodiment includes a method for any of the first through twenty third enumerated embodiments further including: setting a desired time interval for updated the baseline image frame to a new image frame; overriding the update of a new baseline image frame if certain safety conditions are met.

A twenty fifth enumerated embodiment includes a method for any of the first through twenty fourth enumerated embodiments further including: performing a collision detection check for a clear line-of-sight UV light path; performing a failsafe checklist prior to commanding a UV irradiation, if the checklist is determined to be safe.

A twenty sixth enumerated embodiment includes a method for any of the first through twenty fifth enumerated embodiments further including: determining a model for a given venue that accounts for a desired energy density to occur over a desired period of time within the venue; determining the power of the available UV sources that could provide UV light to disinfect an area; determine the time and intensity for each UV source to achieve a desired level of energy density; and keeping track of previous amounts of UV light placed in that area, if multiple passes are desired to reach a given energy density.

A twenty seventh enumerated embodiment includes a method for any of the first through twenty sixth enumerated embodiments further including: affecting a duty cycle of disinfection for desired amounts of airspace and surfaces in a venue; keeping track of the amount of energy density applied to a given area; monitor area for interruptions in affecting the duty cycle; and continue to affect the duty cycle once the interruption has been cleared.

A twenty eighth enumerated embodiment includes a method for any of the first through twenty seventh enumerated embodiments further including: determining blocks in an image frame that indicate motion; ensuring no UV light is sent to those blocks; setting a time period during which no UV light is to be sent to those blocks; determining whether the system predicts that object in the block is living, within that time period; continuing to discontinue UV irradiation if there is a desired probability that the object is predicted to be living; and continuing to discontinue UV irradiation if there is a IR sensor that detect that the object has a heat signature that is consistent with a living being.

A twenty ninth enumerated embodiment includes a method for any of the first through twenty eighth enumerated embodiments further including: determining an area that has a heat signature that may correlate with the head of a person; modeling an area where the body of that person's head may be in the area; and discontinue UV irradiation in the area where the body of the person is modeled.

A thirtieth enumerated embodiment includes a method for any of the first through twenty ninth enumerated embodiments further including: creating a histogram of movement changes in a venue; noting the areas with the highest movement changes over time; scheduling a disinfection of the areas with the highest movement changes.

A thirty first enumerated embodiment includes a method for any of the first through thirtieth enumerated embodiments further including: placing a set of markers throughout the venue; placing at least one sensor throughout the venue such that the sensor can detect the set of markers; commanding a UV irradiation of the area where markers are detected and not obstructed.

A thirty second enumerated embodiment includes a method for any of the first through thirty first enumerated embodiments further including: placing a set of markers into UV absorptive material throughout the venue; directing UV light into those UV absorptive materials when the markers are detected and not obstructed.

A thirty third enumerated embodiment includes a method for any of the first through thirty second enumerated embodiments further including: placing a set of markers at various heights within a venue; commanding UV light to irradiate areas where the markers are detected and not obstructed; commanding UV light go to an area of different height if one area at a given height is obstructed.

A thirty fourth enumerated embodiment includes a method for any of the first through thirty third enumerated embodiments further including: placing a set of UV activated phosphors throughout a venue; directing UV light to irradiate areas that are not obstructed that have the phosphors; detecting the level of light emitted from the phosphors; detecting an error condition if the level of light emitted from the phosphors is not what is expected from the system.

A thirty fifth enumerated embodiment includes: a UV wall including a set of UV light sources embedded in a strip, wherein the light from adjacent UV light sources provides a substantially overlapping UV light field directed to a terminus area; wherein further the overlapping UV light field provides a partition of at least two areas in the venue in which pathogens are subjected to germicidal radiation if traveling from one area to another area.

A thirty sixth enumerated embodiment including the UV wall of the thirty fifth enumerated embodiment further including: the terminus includes a UV absorptive material to reduce the amount of UV scatter in the venue.

A thirty seventh enumerated embodiment including the UV wall in any of the thirty fifth through thirty sixth enumerated embodiments further including: the terminus includes a set of retro-reflectors to reflect and reuse the UV light irradiating the terminus.

A thirty eighth enumerated embodiment including the UV wall of any of the thirty fifth through thirty seventh enumerated embodiment further including: a set of sensor, the sensors configured to detect when the UV light is obstructed and a controller, the controller receiving the sensor data and instructing the UV lights to turn OFF.

A thirty ninth enumerated embodiment including the UV wall of any of the thirty fifth through thirty eighth enumerated embodiment further including: the strip and terminus are dished structures; a set of UV emitters placed in the strip such that the spread function of the set of UV emitters overlap to provide substantially consistent illumination.

A fortieth enumerated embodiment including the UV wall of any of the thirty fifth through thirty ninth enumerated embodiments further including: the strip and terminus are parabolic structures; and edge reflectors to reflect UV light back into the UV wall.

A forty first enumerated embodiment includes: a light fixture including a set of light sources wherein one of the light sources includes a UV source and a second of the light sources is a visible light source.

A forty second enumerated embodiment including the light fixture the forty first enumerated embodiment further including: the light fixture is placed above a table surface and the light fixture emits UV light under controller signals when people are not at the table.

A forty third enumerated embodiment includes: a UV wall of any of the thirty fifth through thirty ninth enumerated embodiments further including: the UV wall is placed on a medical structure wherein the plane of the UV wall is parallel to the plane of the patient.

A forty fourth enumerated embodiment includes: a UV wall of any of the thirty fifth through thirty ninth enumerated embodiments and forty third enumerated embodiment further including: the UV wall when activated provides a UV bubble to substantially protect the patient and the healthcare worker against transmission of infection.

A forty fifth enumerated embodiment including: a table substrate, the table substrate supporting a top layer of a durable, UV transmissive material, an optional second layer of UV transmissive material, and a third layer of a diffuse UV reflective material.

A forty sixth enumerated embodiment including any of the forty fifth enumerated embodiments further including: the top layer including one of Corning Willow Glass, quartz and mica.

A forty seventh enumerated embodiment including any of the forty fifth through forty sixth enumerated embodiments further including: the optional second layer including a layer of UV transmissive silicone, the optional second layer having a thickness sufficient to allow substantial lateral transmission of UV light that is reflected by the third layer.

A forty eighth enumerated embodiment including any of the forty fifth through forty seventh enumerated embodiments further including: the third layer including one of a roughed reflective metallic film, a white sand, a white pigment and mica.

A forty ninth enumerated embodiment including a UV disinfecting table structure further including a set of individually controllable UV LEDs placed in a table substrate; a set of sensor, the sensors dispersed around and between the set of UV LEDs, an optional UV transmissive material placed over top of the UV LEDs, and a top layer of a UV transmissive durable surface.

A fiftieth enumerated embodiment including any of the forty ninth enumerated embodiments further including: the set of UV LEDs include one of UV-A, UV-B UV-C or VUV LEDs.

A fifty first enumerated embodiment including any of the forty ninth through fiftieth enumerated embodiments further including: the set of sensors detecting ambient visible light and a controller receiving the sensor data; the controller sending out control signals to the LEDs such that when the sensors detect a desired decrease in ambient light, the controller sends signals to neighboring LEDs to turn ON, and such that when the sensor detect a desired increase in ambient light the controller sends signals to neighboring LEDs to turn OFF.

A fifty second enumerated embodiment including a UV disinfecting table structure further including a set of UV lamps embedded in a table structure, an optional UV transmissive layer on top of the UV lamps, a LCD layer on top of the UV lamps and optional UV transmissive layer, a set of sensor around the table surface and a top layer of UV transmissible durable material.

A fifty third enumerated embodiment including any of the fifty second enumerated embodiments further including: the set of sensors detecting ambient visible light and a controller receiving the sensor data; the controller sending out control signals to the LEDs such that when the sensors detect a desired decrease in ambient light, the controller sends signals to neighboring LCD cells to transmit UV light from below, and such that when the sensor detect a desired increase in ambient light the controller sends signals to neighboring LCD cless to stop transmission of UV light.

The present application includes a computer program product which is a storage medium (media) having instructions stored thereon/in which can be used to control, or cause, a computer to perform any of the processes of the present application. The storage medium can include, but is not limited to, any type of disk including floppy disks, mini disks (MD's), optical discs, DVD, HD-DVD, Blue-ray, CD-ROMS, CD or DVD RW+/−, micro-drive, and magneto-optical disks, ROMs, RAMs, EPROMs, EEPROMs, DRAMs, VRAMs, flash memory devices (including flash cards, memory sticks), magnetic or optical cards, SIM cards, MEMS, nanosystems (including molecular memory ICs), RAID devices, remote data storage/archive/warehousing, or any type of media or device suitable for storing instructions and/or data.

Stored on any one of the computer readable medium (media), the present application includes software for controlling both the hardware of the general purpose/specialized computer or microprocessor, and for enabling the computer or microprocessor to interact with a human user or other mechanism utilizing the results of the present application. Such software may include, but is not limited to, device drivers, operating systems, and user applications. Ultimately, such computer readable media further includes software for performing the present application, as described above.

Included in the programming (software) of the general/specialized computer or microprocessor are software modules for implementing the teachings of the present application, including, but not limited to, venue management software running at the venue or on a remote server, object and people recognition modules whether provided through APIs or specifically programmed. The present application includes modification of available software specific to applications of the present application and modification or applications specific to a particular venue such as a specific coffee shop, specific theater, or other specific venue, and any necessary or desirable display, storage, or communication of results according to the processes of the present application.

What is claimed is:

1. A system for continuous disinfecting areas in a populated indoor space, the system comprising:
a ultraviolet (UV) light source, the UV light source comprising one or more of coherent and incoherent light sources emitting one or more wavelengths that disinfect surfaces of pathogens;
a set of sensors, the sensors placed in the space and collecting data correlating to characteristics of a plurality of objects and events in the space;
a controller, the controller comprising a processor and computer memory, the computer memory comprising computer-readable instructions, such that when read by the processor, causes the controller to:
receive data from the set of sensors regarding the data correlating to objects in the space;
identify areas in the space to be disinfected;
detecting whether the UV source has a clear line of sight for disinfecting the space to be disinfected; and
send control signals to the light source to control the application of disinfecting light to areas in the space if there is a clear line of sight.

2. The system of claim 1 wherein further the light source comprises one or more light sources emitting one or more of UV-A, UV-B, UV-C, X-ray, microwave and infrared.

3. The system of claim 1 wherein the UV light source comprises a reflector, the reflector being one of Digital Micromirror Device (DMD), Micro Electro-Mechanical System (MEMS), diffraction grating, Liquid System on Silicon (LCoS).

4. The system of claim 1 wherein the set of sensors comprises one or more of cameras, stereo vision, LiDAR, radar, heat sensors, ultrasonic sensors and audio microphones.

5. The system of claim 1 wherein the computer readable instructions further causes the controller to:
identify objects in the area to be disinfected, the objects comprising surfaces to be disinfected.

6. The system of claim 5 wherein the computer readable instructions further causes the controller to:
identify the location of people in the space.

7. The system of claim 6 wherein the controller sending out control signals further comprises:
implementing one or more cleaning protocols for the space.

8. The system of claim 7 wherein one or more cleaning protocols comprises:
applying disinfecting light to the surfaces of objects identified to be disinfected.
